(19) United States
(12) Patent Application Publication
(10) Pub. No.: US 2021/0177044 A1
(43) Pub. Date: Jun. 17, 2021

(54) ORAL PRODUCT

(71) Applicant: NICOVENTURES TRADING LIMITED, London (GB)

(72) Inventors: Anthony Richard Gerardi, Winston-Salem, NC (US); Thomas H. Poole, Winston-Salem, NC (US); Steven Lee Alderman, Lewisville, NC (US)

(21) Appl. No.: 17/186,749

(22) Filed: Feb. 26, 2021

Related U.S. Application Data
(63) Continuation of application No. PCT/IB2020/061344, filed on Dec. 2, 2020.
(60) Provisional application No. 62/945,493, filed on Dec. 9, 2019.

(51) Int. Cl. A24B 15/30; A24B 15/24; A24F 23/02

(52) U.S. Cl. CPC A24B 15/303; A24F 23/02; A24B 15/241; A24B 15/301

(57) ABSTRACT

The disclosure provides products configured for oral use and a process for preparing such oral products. The products include a cannabinoid, a filler, and water. The water content of the oral product is at least about 10% by weight of the oral product, and the water activity of the oral product is no greater than about 0.85.

### CROSS-REFERENCE TO RELATED APPLICATIONS

This application is a continuation of International Application No. PCT/IB2020/061344, filed Dec. 2, 2020, and claims priority to U.S. Provisional Application No. 62/945,493, filed on Dec. 9, 2019, which are incorporated herein by reference in their entirety and for all purposes.

### FIELD

The present disclosure relates to an oral product, a method of making the oral product, as well as pouched products and packages comprising said oral product. In particular, the present disclosure relates to products intended for human use. The products are configured for oral use and deliver an active ingredient during use. Such products include a cannabinoid or a product derived from a cannabinoid.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Smokeless tobacco product configurations that combine tobacco material with various binders and fillers have been proposed more recently, with example product formats including lozenges, pastilles, gels, extruded forms, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

All-white snus portions are growing in popularity, and offer a discrete and aesthetically pleasing alternative to traditional snus. Such modern “white” pouched products may include a bleached tobacco or may be tobacco-free.

It would be desirable to provide products configured for oral use which may deliver active ingredients to the consumer in an enjoyable form, such as in the form of a pouched product.

### BRIEF SUMMARY

In accordance with some embodiments described herein, there is provided an oral product comprising (i) a cannabinoid; (ii) a filler; and (iii) water; wherein the water content of the oral product is at least about 10% by weight of the oral product, and wherein the water activity of the oral product is no greater than about 0.85.

In accordance with some embodiments described herein, there is provided a pouched oral product comprising a saliva permeable pouch and an oral product as defined herein incorporated within the pouch, wherein the water activity of the oral product is no greater than about 0.85.

In accordance with some embodiments described herein, there is provided a package containing an oral product as defined herein or at least one pouched oral product as defined herein.

In accordance with some embodiments described herein, there is provided a process for preparing an oral product as defined herein, the process comprising:

(a) providing a cannabinoid, a filler and water, and

(b) contacting the cannabinoid, the filler and the water to form an oral product;

wherein the water activity of the oral product is no greater than about 0.85.

The disclosure includes, without limitations, the following embodiments.

Embodiment 1: An oral product comprising: (i) a cannabinoid; (ii) a filler; and (iii) water; wherein the water content of the oral product is at least about 10% by weight of the oral product, and wherein the water activity of the oral product is no greater than about 0.85.

Embodiment 2: An oral product according to embodiment 1, wherein the water content of the oral product is from about 10% to about 30% by weight of the oral product.

Embodiment 3: An oral product according to embodiment 1 or 2, wherein the filler comprises a cellulose material selected from the group consisting of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, derivatives thereof, and combinations thereof.

Embodiment 4: An oral product according to embodiment 3, wherein the cellulose material is a derivative of wood pulp fiber.

Embodiment 5: An oral product according to embodiment 4, wherein the cellulose material is microcrystalline cellulose.

Embodiment 6: An oral product according to any one of embodiments 1 to 5, wherein the filler is present in an amount of at least about 50% by weight of the oral product.

Embodiment 7: An oral product according to any one of embodiments 1 to 6, wherein the filler is present in an amount of from about 55% to about 95% by weight of the oral product.

Embodiment 8: An oral product according to any one of embodiments 1 to 7, wherein the cannabinoid is present in an amount of from about 1% to about 30% by weight of the oral product.

Embodiment 9: An oral product according to any one of embodiments 1 to 8, wherein the cannabinoid is present in an amount of from about 5% to about 15% by weight of the oral product.

Embodiment 10: An oral product according to any one of embodiments 1 to 9, wherein the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

Embodiment 11: An oral product according to any one of embodiments 1 to 10, wherein the cannabinoid comprises cannabidiol.

Embodiment 12: An oral product according to embodiment 11, wherein the cannabinoid comprises cannabidiol in an amount of at least 98% by weight of the cannabinoid.

Embodiment 13: An oral product according to any one of embodiments 1 to 12, wherein the oral product contains an emulsion comprising a continuous phase and a dispersed phase, wherein the emulsion comprises at least one cannabinoid.

Embodiment 14: An oral product according to any one of embodiments 1 to 13, wherein the oral product further comprises one or more emulsifying agents.

Embodiment 15: An oral product according to embodiment 13 or 14, wherein the emulsion is in the form of a nanoemulsion in which nanoparticles of an oil phase are dispersed in an aqueous phase.

Embodiment 16: An oral product according to embodiment 15, wherein the cannabinoid is present in the nanoparticles of the oil phase.

Embodiment 17: An oral product according to any one of embodiments 1 to 16, wherein the oral product further comprises at least one additive selected from the group consisting of a flavoring agent, a taste modifier, a preservative, a humectant, a sweetener, a binder, a buffering agent, salt and mixtures thereof.

Embodiment 18: An oral product according to any one of embodiments 1 to 17, wherein the oral product comprises at least one humectant.

Embodiment 19: An oral product according to embodiment 18, wherein the humectant is selected from the group consisting of glycerine, 1,2-propanediol, 1,3-propanediol, dipropylene glycol, sorbitol, xylitol, maltitol, and mixtures thereof.

Embodiment 20: An oral product according to embodiment 18 or 19, wherein the humectant is present in an amount of from about 0.1% to about 20% by weight of the oral product.

Embodiment 21: An oral product according to any one of embodiments 17 to 20, wherein the oral product comprises salt.

Embodiment 22: An oral product according to any one of embodiments 1 to 21, wherein the oral product is chemically and physically stable for a period of at least 6 months.

Embodiment 23: An oral product according to any one of embodiments 1 to 22, wherein at least 50 wt % of the cannabinoid is released within at most about 60 minutes when placed in the oral cavity of a user.

Embodiment 24: An oral product according to any one of embodiments 1 to 23, wherein at least 30 wt % of the released cannabinoid is absorbed into the oral mucosa within at most about 60 minutes.

Embodiment 25: A pouched oral product comprising a saliva permeable pouch and an oral product as defined in any one of embodiments 1 to 24 incorporated within the pouch, wherein the water activity of the oral product is no greater than about 0.85.

Embodiment 26: A package containing an oral product as defined in any one of embodiments 1 to 24 or at least one pouched oral product as defined in embodiment 25.

Embodiment 27: A process for preparing an oral product as defined in any one of embodiments 1 to 24, the process comprising: (a) providing a cannabinoid, a filler and water, and (b) contacting the cannabinoid, the filler and the water to form an oral product; wherein the water activity of the oral product is no greater than about 0.85.

Embodiment 28: A product, package, or process according to any one of embodiments 1 to 27, wherein the cannabinoid is replaced in whole or in part with a cannabimimetic.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are exemplary only, and should not be construed as limiting the disclosure. Embodiments of the invention will now be described, by way of example only, with reference to accompanying drawings, in which:

FIG. 1 is a cross-sectional view of a pouched product embodiment, taken across the width of the product, showing an outer pouch filled with a composition of the present disclosure.

### DETAILED DESCRIPTION

As described herein, there is provided an oral product comprising (i) a cannabinoid; (ii) a filler; and (iii) water; wherein the water content of the oral product is at least about 10% by weight of the oral product, and wherein the water activity of the oral product is no greater than about 0.85.

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the composition including water. Unless otherwise indicated, reference to “weight percent” of a composition reflects the total wet weight of the composition (i.e., including water).

The oral product as described herein comprises a cannabinoid, a filler and water. The relative amounts of the various components within the product may vary, and typically are selected so as to provide the desired sensory and performance characteristics to the oral product. The example individual constituents of the composition are described herein below.

### Oral Product

The oral product is configured for oral use, and thus for insertion into the user's mouth (i.e., oral cavity).

### Filler

As described herein, the oral product includes a filler. Fillers may fulfil multiple functions, such as enhancing certain organoleptic properties such as texture and mouthfeel, enhancing cohesiveness or compressibility of the product, and the like, depending on the product and the association between the filler and the active agent.

In some embodiments, the filler is a porous particulate material and is cellulose-based. For example, the filler may be a non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof.

In some embodiments, the filler is a cellulose material selected from the group consisting of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, derivatives thereof, and combinations thereof. In some embodiments, the filler is a cellulose material selected from the group consisting of maize fiber, oat fiber, sugar beet fiber, bamboo fiber, wood pulp fiber, cotton fiber, grass fiber, derivatives thereof, and combinations thereof. In some embodiments, the filler is a cellulose material selected from the group consisting of sugar beet fiber, wood pulp fiber, bamboo fiber, derivatives thereof, and combinations thereof.

In some embodiments, the filler is derived from any of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, or combinations thereof. In some embodiments, the filler is derived from wood pulp fiber.

In some embodiments, the filler is a cellulose material. One particularly suitable filler for use in the compositions described herein is microcrystalline cellulose (“MCC”). MCC is typically derived from wood pulp fiber. MCC is composed of glucose units connected by a 1-4 beta glycosidic bond, and may be synthesized by partially depolymerizing alpha-cellulose, by, for example, reactive extrusion, enzyme mediated depolymerisation, mechanical grinding, ultrasonication, steam explosion and/or acid hydrolysis. The MCC may be synthetic or semi-synthetic, or it may be obtained entirely from natural celluloses. The MCC may be selected from the group consisting of AVICEL® grades PH-100, PH-101, PH-102, PH-103, PH-105, PH-112, PH-113, PH-200, PH-300, PH-301, PH-302, VIVACEL® grades 101, 102, 12, 20 and EMOCEL® grades 50M and 90M, and the like, and mixtures thereof. In some embodiments, the oral product comprises MCC as the filler.

In some embodiments, the filler is a non-tobacco plant material or a derivative thereof. Non-limiting examples of derivatives of non-tobacco plant material include starches (e.g., from potato, wheat, rice, corn), natural cellulose, and modified cellulosic materials. Additional examples of potential fillers include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the composition based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

The amount of the filler can vary, but when present, is typically at least about 50 percent by weight of the oral product, based on the total weight of the oral product. A typical range of filler (e.g., a cellulose material, such as microcrystalline cellulose) within the composition can be from about 10 to about 75 percent by total weight of the oral product. For example, the filler (e.g., MCC) may be present in the oral product in an amount of at least about 50% by weight of the oral product, such as at least about 55% by weight of the oral product, such as at least about 60% by weight of the oral product. In some embodiments, the filler (e.g., MCC) may be present in the oral product in an amount of from about 50% to about 99% by weight of the oral product, such as from about 50% to about 95% by weight of the oral product, such as from about 50% to about 90% by weight of the oral product, such as from about 55% to about 85% by weight of the oral product, such as from about 60% to about 80% by weight of the oral product, such as from about 60% to about 75% by weight of the oral product.

In some embodiments, the oral product comprises microcrystalline cellulose in an amount of from about 55% to about 95% by weight of the oral product. In some embodiments, the oral product comprises microcrystalline cellulose in an amount of from about 55% to about 80% by weight of the oral product.

It has been surprisingly found that, when the filler is included in the oral product in an amount of at least about 50% by weight of the oral product, such as at least about 55% by weight of the oral product, the shelf-life of the oral product may be improved. For example, the extent of microbiological growth on the product may be reduced over a certain period of time.

### Active Ingredient

As described herein, the oral product comprises at least one cannabinoid.

Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof. In some embodiments, the cannabinoid comprises at least tetrahydrocannabinol (THC). In some embodiments, the cannabinoid is tetrahydrocannabinol (THC). In some embodiments, the cannabinoid comprises at least cannabidiol (CBD). In some embodiments, the cannabinoid is cannabidiol (CBD).

In some embodiments, the cannabinoid is cannabidiol (CBD) or a pharmaceutically acceptable salt thereof. In some embodiments, the cannabidiol is synthetic cannabidiol. In some embodiments, the cannabinoid is added to the oral product in the form of an isolate. In some embodiments, the cannabidiol is added to the oral product in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the oral product is no greater than about 1% by weight of the oral product, such as no greater than about 0.5% by weight of the oral product, such as no greater than about 0.1% by weight of the oral product, such as no greater than about 0.01% by weight of the oral product.

The choice of cannabinoid and the particular percentages thereof which may be present within the disclosed oral product will vary depending upon the desired flavor, texture, and other characteristics of the oral product.

In some embodiments, the cannabinoid (such as cannabidiol) is present in the oral product in a concentration of at least about 0.001% by weight of the oral product, such as in a range from about 0.001% to about 20% by weight of the oral product. In some embodiments, the cannabinoid (such as cannabidiol) is present in the oral product in a concentration of from about 0.1% to about 15% by weight, based on the total weight of the oral product. In some embodiments, the cannabinoid (such as cannabidiol) is present in a concentration from about 1% to about 15% by weight, such as from about from about 5% to about 15% by weight, based on the total weight of the oral product. In some embodiments, the cannabinoid (such as cannabidiol) is present in the oral product in a concentration of from about 0.5% to about 10% by weight, such as from about 1% to about 7.5% by weight, such as from 1.5% to about 5% by weight, such as from about 1.5% to about 2.5% by weight, based on the total weight of the oral product.

As described herein, the cannabinoid is present in the oral product in combination with a filler. The cannabinoid as disclosed herein may be associated with the filler (such as cellulose material) in various ways. For example, the cannabinoid may be disposed on the surface of a filler (such as a cellulose material, such as microcrystalline cellulose), may be dispersed in or impregnated into (e.g., adsorbed or absorbed) the filler, or the filler and the cannabinoid may be present in an oral product without being physically combined or in physical contact (e.g., they may be provided separately and independently within the same product). In some embodiments, the cannabinoid is dispersed in or impregnated into (e.g., adsorbed or absorbed) microcrystalline cellulose. For example, the cannabinoid may be retained within the pores of the microcrystalline cellulose. In some embodiments, the cannabinoid may be disposed on the surface of microcrystalline cellulose.

In some embodiments, the weight ratio of filler (such as microcrystalline cellulose) to cannabinoid is from about 5:1 to about 100:1, such as from about 10:1 to about 60:1, such as from about 15:1 to about 50:1, such as from about 20:1 to about 40:1, such as from about 25:1 to about 35:1. In some embodiments, the weight ratio of microcrystalline cellulose to cannabidiol is from about 5:1 to about 100:1, such as from about 10:1 to about 60:1, such as from about 15:1 to about 50:1, such as from about 20:1 to about 40:1, such as from about 25:1 to about 35:1.

Alternatively, or in addition to the cannabinoid, the oral product can include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

In some embodiments, the oral product may comprise a further active ingredient in combination with the cannabinoid. In some embodiments, two or more active ingredients can be incorporated within the same oral product. For example, the oral product may include one or more active ingredients in addition to the cannabinoid.

As used herein, an “active ingredient” refers to one or more substances belonging to any of the following categories: API (active pharmaceutical substances), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods”. These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients (e.g., hemp, lavender, peppermint, eucalyptus, rooibos, fennel, cloves, chamomile, basil, rosemary, clove, citrus, ginger, cannabis, ginseng, maca, and tisanes), stimulants (e.g., caffeine or guarana), amino acids (e.g., taurine, theanine, phenylalanine, tyrosine, and tryptophan), vitamins e.g., (B6, B12, and C), antioxidants, nicotine components, pharmaceutical ingredients (e.g., nutraceutical and medicinal ingredients), and/or melatonin. Each of these categories is further described herein below. The particular choice of active ingredients will vary depending upon the desired flavor, texture, and desired characteristics of the particular product.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided herein below.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species).

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include hemp, eucalyptus, rooibos, fennel, citrus, cloves, lavender, peppermint, chamomile, basil, rosemary, ginger, turmeric, green tea, white mulberry, cannabis, cocoa, ashwagandha, baobab, chlorophyll, cordyceps, damiana, ginseng, guarana, and maca. In some embodiments, the composition comprises green tea, turmeric, and white mulberry.

Ashwagandha (Withania somnifera) is a plant in the Solanaceae (nightshade) family. As an herb, Ashwagandha has found use in the Indian Ayurvedic system of medicine, where it is also known as “Indian Winter cherry” or “Indian Ginseng.” In some embodiments, the active ingredient comprises ashwagandha.

Baobab is the common name of a family of deciduous trees of the genus Adansonia. The fruit pulp and seeds of the Baobab are consumed, generally after drying, as a food or nutritional supplement. In some embodiments, the active ingredient comprises baobab.

Chlorophyll is any of several related green pigments found in the mesosomes of cyanobacteria, as well as in the chloroplasts of algae and plants. Chlorophyll has been used as a food additive (colorant) and a nutritional supplement. Chlorophyll may be provided either from native plant materials (e.g., botanicals) or in an extract or dried powder form. In some embodiments, the active ingredient comprises chlorophyll.

Cordyceps is a diverse genus of ascomycete (sac) fungi which are abundant in humid temperate and tropical forests. Members of the cordyceps family are used extensively in traditional Chinese medicine. In some embodiments, the active ingredient comprises cordyceps.

Damiana is a small, woody shrub of the family Passifloraceae. It is native to southern Texas, Central America, Mexico, South America, and the Caribbean. Damiana produces small, aromatic flowers, followed by fruits that taste similar to figs. The extract from damiana has been found to suppress aromatase activity, including the isolated compounds pinocembrin and acacetin. In some embodiments, the active ingredient comprises damiana.

Guarana is a climbing plant in the family Sapindaceae, native to the Amazon basin. The seeds from its fruit, which are about the size of a coffee bean, have a high concentration of caffeine and, consequently, stimulant activity. In some embodiments, the active ingredient comprises guarana. In some embodiments, the active ingredient comprises guarana, honey, and ashwagandha.

Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the active ingredient comprises ginseng. In some embodiments, the ginseng is American ginseng or Korean ginseng. In specific embodiments, the active ingredient comprises Korean ginseng.

Maca is a plant that grows in central Peru in the high plateaus of the Andes Mountains. It is a relative of the radish, and has an odor similar to butterscotch. Maca has been used in traditional (e.g., Chinese) medicine. In some embodiments, the active ingredient comprises maca.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

In some embodiments, the active ingredient comprises caffeine. In some embodiments, the active ingredient comprises theacrine. In some embodiments, the active ingredient comprises a combination of caffeine and theacrine.

### Amino Acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-tranlational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine.

When present, an amino acid or combination of amino acids (e.g., taurine, theanine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

In some embodiments, the amino acid is taurine, theanine, phenylalanine, tyrosine, tryptophan, or a combination thereof. In some embodiments, the amino acid is taurine. In some embodiments, the active ingredient comprises a combination of taurine and caffeine. In some embodiments, the active ingredient comprises a combination of taurine, caffeine, and guarana. In some embodiments, the active ingredient comprises a combination of taurine, maca, and cordyceps. In some embodiments, the active ingredient comprises a combination of theanine and caffeine.

### Vitamins

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones).

When present, a vitamin or combination of vitamins (e.g., vitamin B6, vitamin B12, vitamin E, vitamin C, or a combination thereof) is typically at a concentration of from about 0.01% w/w to about 1% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, or about 1% by weight, based on the total weight of the composition.

In some embodiments, the vitamin is vitamin B6, vitamin B12, vitamin E, vitamin C, or a combination thereof. In some embodiments, the active ingredient comprises a combination of vitamin B6, caffeine, and theanine. In some embodiments, the active ingredient comprises vitamin B6, vitamin B12, and taurine. In some embodiments, the active ingredient comprises a combination of vitamin B6, vitamin B12, ginseng, and theanine. In some embodiments, the active ingredient comprises a combination of vitamin C, baobab, and chlorophyll.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage.

Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, Bacopa monniera, Withania somnifera, Lion's mane, and Silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12 (2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrol s, oleuropein, butylated hydroxyanisole (BHA), butylated hydroxytoluene (BHT), tertiary butylhydroquinone (TBHQ), and combinations thereof. In some embodiments, the antioxidant is Vitamin E or a derivative thereof, a flavonoid, a polyphenol, a carotenoid, or a combination thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the composition.

### Nicotine Component

In certain embodiments, a nicotine component may be further included in the oral product. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, nicotine is in its free base form, which can be easily adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Further salts are disclosed in, for example, U.S. Pat. No. 9,738,622 to Dull et al., and US Pat. Pub. Nos. 2018/0230126 to Dull et al., 2016/0185750 to Dull et al., and 2018/0051002 to Dull et al., each of which is incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the oral product, such as in a range from about 0.001% to about 10%. In some embodiments, the nicotine component is present in a concentration from about 0.1% to about 10% by weight, such as from about from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10%, calculated as the free base and based on the total weight of the oral product. In some embodiments, the nicotine component is present in a concentration from about 0.1% to about 3% by weight, such as from about from about 0.1% to about 2.5%, such as from about 0.1% to about 2.0%, such as from about 0.1% to about 1.5%, such as from about 0.1% to about 1% by weight, calculated as the free base and based on the total weight of the oral product. These ranges can also apply to other additional active ingredients noted herein.

In some embodiments, the oral product of the disclosure can be characterized as completely free or substantially free of nicotine. For example, certain embodiments can be characterized as having less than 0.1% by weight, or less than 0.01% by weight, or less than 0.001% by weight of a nicotine component, or 0% by weight of a nicotine component, based on the total weight of the oral product.

### Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Pharmaceutical Ingredients

The pharmaceutical ingredient can be any known agent adapted for therapeutic, prophylactic, or diagnostic use. These can include, for example, synthetic organic compounds, proteins and peptides, polysaccharides and other sugars, lipids, inorganic compounds, and nucleic acid sequences, having therapeutic, prophylactic, or diagnostic activity. Non-limiting examples of pharmaceutical ingredients include analgesics and antipyretics (e.g., acetylsalicylic acid, acetaminophen, 3-(4-isobutylphenyl)propanoic acid).

### Water

As described herein, the oral product comprises water, wherein the water content of the oral product is at least about 10% by weight of the oral product. As referred to herein, “the water content” means the total amount of water in the oral product, as included in any form. Water may be present as, for example, purified or ultrapure water, saline, buffered saline, or a buffered aqueous phase.

In some embodiments, the oral product has a water content of at least about 11% by weight, such as at least about 12% by weight, such as at least about 13% by weight, such as at least about 14% by weight, such as at least about 15% by weight of the oral product.

In some embodiments, the oral product has a water content of from about 10% to about 30% by weight of the oral product, such as from about 10% to about 25% by weight of the oral product, such as from about 10% to about 20% by weight of the oral product, such as from about 11% to about 15% by weight of the oral product. In some embodiments, the oral product has a water content of from about 12% to about 30% by weight of the oral product, such as from about 13% to about 25% by weight of the oral product, such as from about 14% to about 25% by weight of the oral product, such as from about 15% to about 20% by weight of the oral product.

In some embodiments, the weight ratio of filler to water is from about 1:1 to about 20:1, such as from about 1:1 to about 10:1, such as from about 2:1 to about 5:1, such as from about 3:1 to about 5:1.

In some embodiments, the weight ratio of water to cannabinoid is from about 1:2 to about 15:1, such as from about 1:1 to about 10:1, such as from about 2:1 to about 8:1, such as from about 3:1 to about 5:1.

As described herein below, the oral product may comprise an emulsion. The emulsion may comprise an oil phase and an aqueous phase. In some embodiments, the only water present in the composition is contained within an emulsion in the product.

It was surprisingly found by the present inventors that a product having a water content of at least 10% by weight but in which the overall water activity is no greater than about 0.85 imparts a desirable mouth-feel to the user, whilst having an acceptable shelf-life. Inclusion of water in an amount of at least 10% by weight provides the user with an oral product that has desirable physical and sensorial properties, and has the mouthfeel of a moist snus type product which is desirable for the average user. That said, by reducing the water activity such that it is no greater than about 0.85, the inventors found that the stability of the product against microbiological growth during storage was improved.

In some embodiments, the shelf-life of the product may be at least about 6 weeks, such as at least about 8 weeks, such as at least about 10 weeks, such as at least about 12 weeks. In some embodiments, the shelf-life of the product may be at least about 6 months. As described herein, the “shelf-life” refers to the period of time during which no visible microbiological growth is observed on the product, and there is no deterioration in the appearance and/or taste of the oral product.

It was also found that the oral product may be both chemically and physically stable for a period of at least 6 months, for example at a relative humidity of 50%. By “chemically and physically stable”, it is understood that the cannabinoid does not migrate out of the product as such a migration will lead to a marked loss of the cannabinoid in the product (chemical stability), and also that no visible changes are observed over the measured period (physical stability) and the dissolution profile when the oral product is inserted into the mouth of a user does not change.

### Further Components

In some embodiments, the oral product may further comprise at least one additive selected from the group consisting of a flavoring agent (or “flavorant”), a taste modifier, a preservative, a humectant, a sweetener, a binder, a buffering agent, salt, and mixtures thereof. The additive(s) may be present in any form within the oral product. For example, in embodiments in which the oral product includes an emulsion as described herein below, the additive(s) may be present in the emulsion or within the oral product separate from the emulsion (e.g., in a mixture with a filler (such as cellulose material) or the like).

### Flavoring Agent and Taste Modifier

In some embodiments, the oral product further comprises a flavorant. As used herein, the terms “flavor” and “flavorant” refer to materials which, where local regulations permit, may be used to create a desired taste, aroma or other somatosensorial sensation in a product for adult consumers. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

They may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavor comprises menthol, spearmint and/or peppermint. In some embodiments, the flavor comprises flavor components of cucumber, blueberry, citrus fruits and/or redberry. In some embodiments, the flavor comprises eugenol. In some embodiments, the flavor comprises flavor components extracted from tobacco. In some embodiments, the flavor comprises flavor components extracted from cannabis.

In some embodiments, the flavor may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

In some embodiments, the flavorant is lipophilic. Without wishing to be bound by theory, formulation of a lipophilic flavorant as an emulsion may enhance the stability of the flavorant (e.g., toward oxidation or evaporation). In some embodiments, the flavorant is susceptible to oxidation, meaning exposure to air results in the degradation of components in the flavorant due to chemical changes. Examples of functional groups which may be present in flavorant components exhibiting susceptibility to oxidation include, but are not limited to, alkenes, aldehydes, and/or ketones. In some embodiments, the flavorant comprises a citrus oil. Citrus oils contain, for example, terpene components which may be susceptible to oxidation, evaporation, or both and, thus, may particularly benefit from inclusion within a product in the form of an emulsion as described herein below.

In some embodiments, the flavoring agent may comprise a terpene. In some embodiments, the terpene is a terpene derivable from a phytocannabinoid producing plant, such as a plant from the stain of the cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the oral product comprises more than one terpene. For example, the oral product may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

The amount of flavorant utilized in the oral product can vary, but is typically up to about 10% by weight, and certain embodiments are characterized by a flavoring agent content of at least about 0.1% by weight, such as about 0.5% to about 10% by weight, about 1 to about 6% by weight, or about 2% to about 5% by weight, based on the total weight of the oral product.

In some embodiments, the oral product comprises a taste modifying agent (or “taste modifier”). In some embodiments, the taste modifier may mask the bitterness of the cannabinoid in the product. The taste modifying agent may improve the organoleptic properties of an oral product as disclosed herein, and may serve to mask, alter, block, or improve e.g., the flavor of a composition as described herein. Non-limiting examples of such taste modifiers include analgesic or anesthetic herbs, spices, and flavors which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the cannabinoid has a bitter taste, and the oral product comprises a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., the cannabinoid). In some embodiments, the taste modifier is capsaicin.

In some embodiments, the taste modifier is the amino acid gamma-amino butyric acid (GABA), referenced herein above with respect to amino acids. Studies in mice suggest that GABA may serve function(s) in taste buds in addition to synaptic inhibition. See, e.g., Dvoryanchikov et al., J Neurosci. 2011 Apr. 13; 31(15):5782-91. Without wishing to be bound by theory, GABA may suppress the perception of certain tastes, such as bitterness. In some embodiments, the composition comprises caffeine and GABA.

In some embodiments, the taste modifier is adenosine monophosphate (AMP). AMP is a naturally occurring nucleotide substance which can block bitter food flavors or enhance sweetness. It does not directly alter the bitter flavor, but may alter human perception of “bitter” by blocking the associated receptor.

In some embodiments, the taste modifier is lactisole. Lactisole is an antagonist of sweet taste receptors. Temporarily blocking sweetness receptors may accentuate e.g., savory notes.

When present, a representative amount of taste modifier is about 0.01% by weight or more, about 0.1% by weight or more, or about 1.0% by weight or more, but will typically make up less than about 10% by weight of the total weight of the oral product, (e.g., from about 0.01%, about 0.05%, about 0.1%, or about 0.5%, to about 1%, about 5%, or about 10% by weight of the total weight of the oral product).

In some embodiments, the taste modifier selected from the group consisting of an analgesic or anesthetic herb, spice, or flavor which produces a perceived cooling or warming effect, gamma-aminobutyric acid, capsaicin, and adenosine monophosphate. In some embodiments, the taste sensation modified by the taste modifier is bitterness, sweetness, saltiness, or sourness. In some embodiments, the taste sensation is bitterness. In some embodiments, the taste modifier is capsaicin.

### Humectant

In certain embodiments, one or more humectants may be employed in the oral product of the present disclosure. The humectant may be present in an emulsion contained within the oral product, or may be present in the composition separate from an emulsion. In some embodiments, the oral product comprises a humectant. Examples of humectants include, but are not limited to, glycerine, 1,2-propanediol (propylene glycol), 1,3-propanediol, dipropylene glycol, sorbitol, xylitol, mannitol, and the like. In some embodiments, the oral product comprises a humectant selected from the group consisting of glycerine, propylene glycol, 1,3-propanediol, dipropylene glycol, sorbitol, xylitol, mannitol, and mixtures thereof. In some embodiments, the oral product comprises a humectant selected from the group consisting of glycerine, propylene glycol, and mixtures thereof.

In some embodiments, the humectant is or comprises glycerine. In some embodiments, the oral product comprises glycerine. In some embodiments, the humectant is or comprises propylene glycol. In some embodiments, the oral product comprises propylene glycol.

Where included, the humectant is typically provided in an amount sufficient to provide desired moisture attributes to the composition. Further, in some instances, the humectant may impart desirable flow characteristics to the composition for depositing in a mold. It has also been found that the inclusion of a humectant, such as glycerine and/or propylene glycol, in the oral product may reduce the overall water activity of the oral product, and thus further improve the stability and shelf-life of the product.

When present in the oral product, the humectant (such as glycerine and/or propylene glycol) may be present in an amount of from about 0.01% to about 25% by weight of the oral product, such as from about 0.1% to about 20% by weight of the oral product, such as from about 0.5% to about 15% by weight of the oral product, such as from about 1% to about 10% by weight of the oral product, such as from about 5% to about 10% by weight of the oral product.

In some embodiments, the oral product comprises glycerine in an amount of from about 0.01% to about 25% by weight of the oral product, such as from about 0.1% to about 20% by weight of the oral product, such as from about 0.5% to about 15% by weight of the oral product, such as from about 1% to about 10% by weight of the oral product, such as from about 5% to about 10% by weight of the oral product.

### Sweetener

In order to improve the sensory properties of the oral product according to the disclosure, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, isomaltulose, mannose, galactose, lactose, stevia, honey, and the like. Examples of artificial sweeteners include sucralose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates).

In some embodiments, the sweetener is selected from the group consisting of fructose, sucrose, glucose, maltose, isomaltulose, mannose, galactose, lactose, stevia, honey, sucralose, maltodextrin, saccharin, aspartame, acesulfame K, neotame, erythritol, arabitol, ribitol, isomalt, maltitol, dulcitol, iditol, mannitol, xylitol, lactitol, sorbitol, and mixtures thereof. In some embodiments, the sweetener is selected from the group consisting of sucralose, acesulfame K, aspartame, maltodextrin, mannitol, sucrose, and mixtures thereof. In some embodiments, the sweetener may be sucralose and/or acesulfame K.

When present in the oral product, the sweetener (such as sucralose and/or acesulfame K) may be present in an amount of from about 0.001% to about 5% by weight of the oral product, such as from about 0.01% to about 3% by weight of the oral product, such as from about 0.1% to about 1% by weight of the oral product.

### Binder

A binder (or combination of binders) may be employed in certain embodiments, in amounts sufficient to provide the desired physical attributes and physical integrity to the composition, and binders also often function as thickening or gelling agents. Typical binders can be organic or inorganic, or a combination thereof. Representative binders include cellulose derivatives (e.g., cellulose ethers), povidone, sodium alginate, starch-based binders, pectin, gums, carrageenan, pullulan, zein, and the like, and combinations thereof. In some embodiments, the binder comprises pectin or carrageenan or combinations thereof.

The amount of binder utilized in the composition can vary, but is typically up to about 30% by weight, and certain embodiments are characterized by a binder content of at least about 0.1% by weight, such as from about 1% to about 30% by weight, or about 1% to about 10% by weight, based on the total weight of the oral product.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the oral product.

### Buffering Agent

In certain embodiments, the oral product of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5% based on the weight of the oral product; for example, from about 0.5% to about 5%, such as, e.g., from about 0.75% to about 4%, from about 0.75% to about 3%, or from about 1% to about 2% by weight, based on the total weight of the oral product.

Non-limiting examples of suitable buffers include alkali metals acetates, glycinates, phosphates, glycerophosphates, citrates, carbonates, hydrogen carbonates, borates, or mixtures thereof. In some embodiments, the buffering agent is selected from the group consisting of sodium carbonate, sodium bicarbonate, sodium phosphate, ammonium phosphate, and mixtures thereof.

The oral product according to the disclosure may have any suitable pH. In certain embodiments, the oral product of the present disclosure has a pH of from about 4 to about 7. In certain embodiments, the oral product of the present disclosure has a pH of from about 4 to about 6.5. In certain embodiments, the oral product of the present disclosure has a pH of from about 4.5 to about 7. In certain embodiments, the oral product of the present disclosure has a pH of from about 4.5 to about 6.5. In certain embodiments, the oral product of the present disclosure has a pH of from about 4 to about 6.5. In certain embodiments, the oral product of the present disclosure has a pH of from about 4.5 to about 6. In certain embodiments, the oral product of the present disclosure has a pH of from about 5 to about 6.

The pH of the oral product may be measured by any suitable technique. For example, the pH of the oral product may be measured by contacting 5 grams of oral product with 95 g of water (100 g total) and then mixing for 5 minutes. After mixing the pH of the solution may be measured with a pH probe.

The emulsion according to the disclosure may have any suitable pH. In certain embodiments, the emulsion of the present disclosure has a pH of from about 4 to about 7. In certain embodiments, the emulsion of the present disclosure has a pH of from about 4.5 to about 7. In certain embodiments, the emulsion of the present disclosure has a pH of from about 5 to about 7. In certain embodiments, the emulsion of the present disclosure has a pH of from about 5.5 to about 7. In certain embodiments, the emulsion of the present disclosure has a pH of from about 6 to about 7. In certain embodiments, the emulsion of the present disclosure has a pH of from about 6 to about 6.5.

### Salt

In some embodiments, the oral product according to the disclosure comprises a salt (e.g., an alkali metal salt), typically employed in an amount sufficient to provide desired sensory attributes to the product. It has also been found that the inclusion of a salt, such as sodium chloride, in the oral product may reduce the overall water activity of the oral product, and thus further improve the stability and shelf-life of the product

Non-limiting examples of suitable salts include sodium chloride, potassium chloride, ammonium chloride, flour salt, sodium acetate, sodium citrate, and the like. When present, a representative amount of salt is at least about 0.5% by weight, such as at least about 1% by weight, such as at least about 1.5% by weight. In some embodiments, the oral product may comprise salt in an amount of from about 0.5% to about 10% by weight, such as from about 1% to about 7.5% by weight, such as from about 1.5% to about 5% by weight, based on the total weight of the oral product.

### Other Additives

Other additives can be included in the oral product. For example, the oral product can be processed, blended, formulated, combined, and/or mixed with other materials or ingredients. The additives can be artificial, or can be obtained or derived from herbal or biological sources. Examples of further types of additives include thickening or gelling agents (e.g., fish gelatin), preservatives (e.g., potassium sorbate, sodium benzoate, calcium propionate, and the like), disintegration aids, zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final composition, with an example range of up to about 10% by weight, (e.g., from about 0.1% to about 5% by weight) based on total weight of the oral product.

For example, where present, a preservative (such as potassium sorbate, sodium benzoate, calcium propionate, or the like) can be included in the oral product in an amount of from about 0.001% to about 5% by weight of the oral product, such as from about 0.01% to about 2.5% by weight of the oral product, such as from about 0.05% to about 1% by weight of the oral product. The inclusion of a preservative may serve to decrease the water activity of the oral product, thus further improving the stability and shelf-life of the oral product.

A colorant may be employed in amounts sufficient to provide the desired physical attributes to the oral product according to the present disclosure. Examples of colorants include various dyes and pigments, such as caramel coloring and titanium dioxide. The amount of colorant utilized in the oral product can vary, but when present is typically up to about 3% by weight, such as from about 0.1%, about 0.5%, or about 1%, to about 3% by weight, based on the total weight of the oral product.

The aforementioned additives can be employed together (e.g., as additive formulations) or separately (e.g., individual additive components can be added at different stages involved in the preparation of the final product). Furthermore, the aforementioned types of additives may be encapsulated as provided in the final product or composition. Exemplary encapsulated additives are described, for example, in WO2010/132444 to Atchley, which is incorporated by reference herein.

### Configured for Oral Use

The oral product as described herein is configured for oral use. The term “configured for oral use” as used herein means that the product is provided in a form such that during use, saliva in the mouth of the user causes one or more of the components of the product (e.g., flavoring agents and/or active ingredients) to pass into the mouth of the user. In certain embodiments, the product is adapted to deliver components to a user through mucous membranes in the user's mouth, the user's digestive system, or both, and, in some instances, said component is an active ingredient that can be absorbed through the mucous membranes in the mouth or absorbed through the digestive tract when the product is used.

In some embodiments, the oral product is a solid oral product. The solid products of the present invention are compositions which can substantially sustain their physical shape when unsupported by external means, e.g., packaging etc. Thus, they are considered to be solid, solid like, in solid form or in solid-like form at room temperature. For the avoidance of doubt the solid product must remain substantially solid at up to 30° C.

By solid-like, it is understood that some materials are considered on a day to day basis to be solid, yet over an extremely long period of time, may alter in shape, e.g., amorphous materials such as glass etc. However, they are considered to be solid-like as, for the purpose they fulfil, they are solid.

In some embodiments, the products configured for oral use as described herein are in a solid form. The products may take various forms, including pastilles, gums, lozenges, tablets, and powders. The products may be provided in pouch form in which a solid oral product (e.g., a powder) is incorporated within a pouch.

Certain products configured for oral use are in the form of pastilles. As used herein, the term “pastille” refers to a dissolvable oral product made by solidifying a liquid or gel composition so that the final product is a somewhat hardened solid gel. The rigidity of the gel is highly variable. Certain products can exhibit, for example, one or more of the following characteristics: crispy, granular, chewy, syrupy, pasty, fluffy, smooth, and/or creamy. In certain embodiments, the desired textural property can be selected from the group consisting of adhesiveness, cohesiveness, density, dryness, fracturability, graininess, gumminess, hardness, heaviness, moisture absorption, moisture release, mouthcoating, roughness, slipperiness, smoothness, viscosity, wetness, and combinations thereof. The products of the present disclosure may be dissolvable. As used herein, the terms “dissolve,” “dissolving,” and “dissolvable” refer to compositions having aqueous-soluble components that interact with moisture in the oral cavity and enter into solution, thereby causing gradual consumption of the product. According to one aspect, the dissolvable product is capable of lasting in the user's mouth for a given period of time until it completely dissolves. Dissolution rates can vary over a wide range, from about 1 minute or less to about 60 minutes. For example, fast release compositions typically dissolve and/or release the active substance in about 2 minutes or less, often about 1 minute or less (e.g., about 50 seconds or less, about 40 seconds or less, about 30 seconds or less, or about 20 seconds or less). Dissolution can occur by any means, such as melting, mechanical disruption (e.g., chewing), enzymatic or other chemical degradation, or by disruption of the interaction between the components of the composition. In some embodiments, the product can be meltable as discussed, for example, in US Patent App. Pub. No. 2012/0037175 to Cantrell et al. In other embodiments, the products do not dissolve during the product's residence in the user's mouth.

In some embodiments, the oral product may be in the form of a powder. The powder may be a free-flowing powder. The powder may be contained in loose form within a container, and may thus be used in a form similar to tobacco snuff where the user takes a pinch of powder from the container and places the powder in the oral cavity. Alternatively or additionally, the powder may be incorporated into a moisture-permeable (e.g., saliva-permeable) pouch, similar to a snus-type product. The pouched product may be configured for insertion into the oral cavity of a user; i.e., it may be a pouched oral product.

In some embodiments, the product of the present disclosure is in the form of a pouched oral product. Such a pouched oral product comprises the oral product as described herein, disposed within a moisture-permeable container (e.g., a water-permeable pouch or saliva-permeable pouch). For example, the pouched product may comprise the oral product in a powder form incorporated within the saliva-permeable pouch.

Therefore, according to some embodiments described herein, there is provided a pouched oral product comprising a saliva permeable pouch and an oral product incorporated within the pouch, wherein the oral product comprises (i) a cannabinoid, (ii) a filler, and (iii) water, wherein the water content of the oral product is at least about 10% by weight of the oral product, and wherein the water activity of the oral product is no greater than about 0.85. The oral product incorporated within the pouch may be in the form of a powder, for example.

Such compositions in the moisture-permeable pouch format are typically used by placing one pouch containing the composition in the mouth of a human subject/user. Generally, the pouch is placed somewhere in the oral cavity of the user, for example under the lips, in the same way as moist snuff products are generally used. The pouch preferably is not chewed or swallowed. Exposure to saliva then causes some of the components of the composition therein (e.g., flavoring agents and/or active ingredients) to pass through e.g., the moisture-permeable pouch and provide the user with flavor and satisfaction, and the user is not required to spit out any portion of the composition. After about 10 minutes to about 60 minutes, typically about 15 minutes to about 45 minutes, of use/enjoyment, substantial amounts of the composition have been ingested by the human subject, and the pouch may be removed from the mouth of the human subject for disposal.

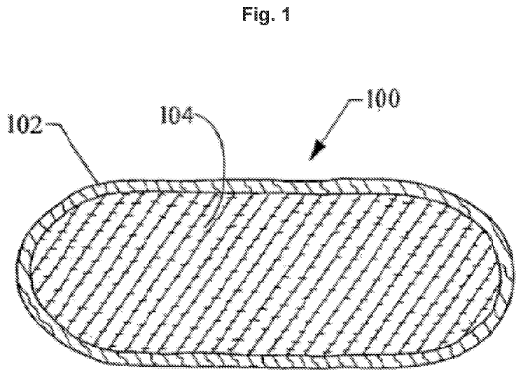

Accordingly, in certain embodiments, the oral product as disclosed herein and any other components noted above are combined within a moisture-permeable packet or pouch that acts as a container for use of the composition to provide a pouched product configured for oral use. Certain embodiments of the disclosure will be described with reference to FIG. 1 of the accompanying drawing, and these described embodiments involve snus-type products having an outer pouch and containing a composition as described herein. As explained in greater detail below, such embodiments are provided by way of example only, and the pouched products of the present disclosure can include the composition in other forms. The composition/construction of such packets or pouches, such as the container pouch 102 in the embodiment illustrated in FIG. 1, may be varied. Referring to FIG. 1, there is shown a first embodiment of a pouched product 100. The pouched product 100 includes a moisture-permeable container in the form of a pouch 102, which contains an oral product 104 that comprises a cannabinoid, a filler and water as described herein.

In some embodiments, the pouch is saliva-permeable. This means that the pouch is made of a saliva-permeable pouch material. In some embodiments, the pouch material is a fleece material. In some embodiments, the pouch material is a non-woven material. In some embodiments, the pouch material is a non-woven fleece material. In some embodiments, the pouch material comprises viscose, such as viscose rayon fibers. In some embodiments, the pouch material comprises regenerated cellulose fibers. In some embodiments, the pouch material comprises polyester fibers; the polyester fibers may constitute the pouch material or may be included in combination with viscose (such as regenerated cellulose fibers).

In some embodiments, the pouch material comprises a binder that provides for heat sealing of the pouches during manufacture. In some embodiments, the pouch material comprises an acrylic binder. In some embodiments, the pouch material comprises an acrylic binder in combination with viscose and/or polyester fibers.

Suitable packets, pouches or containers of the type used for the manufacture of smokeless tobacco products are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. The composition may be contained in pouches and packaged, in a manner and using the types of components used for the manufacture of conventional snus types of products. The pouch provides a moisture-permeable container of a type that may be considered to be similar in character to the mesh-like type of material that is used for the construction of a tea bag. Components of the composition readily diffuse through the pouch and into the mouth of the user.

Non-limiting examples of suitable types of pouches are set forth in, for example, U.S. Pat. No. 5,167,244 to Kjerstad and U.S. Pat. No. 8,931,493 to Sebastian et al.; as well as US Patent App. Pub. Nos. 2016/0000140 to Sebastian et al.; 2016/0073689 to Sebastian et al.; 2016/0157515 to Chapman et al.; and 2016/0192703 to Sebastian et al., each of which is incorporated herein by reference. Pouches can be provided as individual pouches, or a plurality of pouches (e.g., 2, 4, 5, 10, 12, 15, 20, 25 or 30 pouches) can be connected or linked together (e.g., in an end-to-end manner) such that a single pouch or individual portion can be readily removed for use from a one-piece strand or matrix of pouches. The pouch may be formed of a moisture-permeable non-woven fabric, such as viscose for example.

An example pouch may be manufactured from materials, and in such a manner, such that during use by the user, the pouch undergoes a controlled dispersion or dissolution. Such pouch materials may have the form of a mesh, screen, perforated paper, permeable fabric, or the like. For example, pouch material manufactured from a mesh-like form of rice paper, or perforated rice paper, may dissolve in the mouth of the user. As a result, the pouch and composition each may undergo complete dispersion within the mouth of the user during normal conditions of use, and hence the pouch and composition both may be ingested by the user. Other examples of pouch materials may be manufactured using water dispersible film forming materials (e.g., binding agents such as alginates, carboxymethylcellulose, xanthan gum, pullulan, and the like), as well as those materials in combination with materials such as ground cellulosics (e.g., fine particle size wood pulp). Preferred pouch materials, though water dispersible or dissolvable, may be designed and manufactured such that under conditions of normal use, a significant amount of the composition contents permeate through the pouch material prior to the time that the pouch undergoes loss of its physical integrity. If desired, flavoring ingredients, disintegration aids, and other desired components, may be incorporated within, or applied to, the pouch material.

The amount of the oral product contained within each pouched product unit, for example, a pouch, may vary. In some embodiments, the weight of the oral product within each pouch is at least about 50 mg, for example, for example, from about 50 mg to about 2 grams, from about 100 mg to about 1.5 grams, or from about 200 to about 700 mg. In some smaller embodiments, the weight of the oral product within each pouch may be from about 100 mg to about 300 mg. For a larger embodiment, the weight of the material within each pouch may be from about 300 mg to about 700 mg. If desired, other components can be contained within each pouch. For example, at least one flavored strip, piece or sheet of flavored water dispersible or water soluble material (e.g., a breath-freshening edible film type of material) may be disposed within each pouch along with or without at least one capsule. Such strips or sheets may be folded or crumpled in order to be readily incorporated within the pouch. See, for example, the types of materials and technologies set forth in U.S. Pat. No. 6,887,307 to Scott et al. and U.S. Pat. No. 6,923,981 to Leung et al.; and The EFSA Journal (2004) 85, 1-32; which are incorporated herein by reference.

In certain embodiments, one or more active ingredients (including at least a cannabinoid) as described herein are included in the oral product within the pouch, and one or more further active ingredients are disposed in or on the external surface of the pouched product (e.g., on or in the pouch material as disclosed herein). In some embodiments, separate location of the active ingredients may allow differential release profiles (e.g., one active ingredient may be rapidly available to the mouth and/or digestive system, and the other active ingredient may be released more gradually with product use). For example, in some embodiments, the composition (or oral product) within the pouched product may include at least one cannabinoid, and at least one cannabinoid may also be disposed in or on the external surface of the pouched product (e.g., on or in the pouch material as disclosed herein). Alternatively or in addition, at least one cannabinoid may be included in the oral product within the pouch, and at least one further and distinct active agent may be included in or on the external surface of the pouch.

According to some embodiments described herein, there is provided a package containing an oral product as described herein. For example, the package may contain the oral product in solid form, such as in powdered form. In such embodiments, the package may be in the form of a tin or plastic container. Alternatively or additionally, the package may contain the oral product in the form of a lozenge, pastille, tablet, or the like. The package may be in the form of a blister pack, tin or plastic container containing such solid oral dosage forms.

According to some embodiments described herein, there is provided a package containing at least one pouched oral product as described herein. A pouched product as described herein can be packaged within any suitable inner packaging material and/or outer container. See also, for example, the various types of containers for smokeless types of products that are set forth in U.S. Pat. No. 7,014,039 to Henson et al.; U.S. Pat. No. 7,537,110 to Kutsch et al.; U.S. Pat. No. 7,584,843 to Kutsch et al.; U.S. Pat. No. 8,397,945 to Gelardi et al., U.S. Pat. No. D592,956 to Thiellier; U.S. Pat. No. D594,154 to Patel et al.; and U.S. Pat. No. D625,178 to Bailey et al.; US Pat. Pub. Nos. 2008/0173317 to Robinson et al.; 2009/0014343 to Clark et al.; 2009/0014450 to Bjorkholm; 2009/0250360 to Bellamah et al.; 2009/0266837 to Gelardi et al.; 2009/0223989 to Gelardi; 2009/0230003 to Thiellier; 2010/0084424 to Gelardi; and 2010/0133140 to Bailey et al; 2010/0264157 to Bailey et al.; and 2011/0168712 to Bailey et al. which are incorporated herein by reference. For example, the package may be a tin or plastic container which contains a plurality of the pouched oral products.

### Emulsion

In some embodiments, the oral product comprises an emulsion that comprises a continuous phase and a dispersed phase. In some embodiments, the emulsion may comprise at least one cannabinoid. In some embodiments, at least one cannabinoid present in the oral product is contained in an emulsion. In some embodiments, all of the cannabinoid(s) present in the oral product are contained in an emulsion; i.e., the oral product comprises an emulsion, wherein the cannabinoid is contained within the emulsion.

In some embodiments, the oral product thus comprises (i) an emulsion comprising a continuous phase and a dispersed phase, the emulsion further comprising at least one cannabinoid; (ii) a filler, and (iii) water. In some embodiments, at least some of the water contained in the oral product is present as part of the emulsion; e.g., in the aqueous phase. In some embodiments, all of the water in the oral product is contained within the emulsion. Therefore, in some embodiments, the oral product comprises (i) an emulsion comprising an aqueous phase comprising water and an oil phase, the emulsion further comprising at least one cannabinoid; and (ii) a filler.

According to some embodiments, the emulsion may be associated with the filler in various ways (i.e., in an oral product comprising an emulsion as disclosed herein). For example, the emulsion may be disposed on the surface of the filler, may be dispersed in or impregnated into (e.g., adsorbed or absorbed) the filler, or the filler and the emulsion may be present in an oral product without being physically combined or in physical contact (e.g., they may be provided separately and independently within the same product). The filler may be or comprise a cellulose material as described hereinabove, such as microcrystalline cellulose.

### Dispersed and Continuous Phases

In some embodiments, the oral product comprises an emulsion that contains a continuous phase and a dispersed phase. The emulsion may further comprise at least one cannabinoid.

Where present, the emulsion may comprise an oil phase as the continuous phase or the dispersed phase. The emulsion may comprise an aqueous phase as the continuous phase or the dispersed phase. In some embodiments, the emulsion comprises an oil phase as the continuous phase and an aqueous phase as the dispersed phase (i.e., a water-in-oil emulsion). In some embodiments, the emulsion comprises an aqueous phase as the continuous phase and an oil phase as the dispersed phase (i.e., an oil-in-water emulsion). In some embodiments, the emulsion may be a water-in-oil-in-water emulsion. In some embodiments, the emulsion may be an oil-in-water-in-oil emulsion.

Any suitable oil may be used to form the emulsion as disclosed herein, including petroleum-based (e.g., mineral oil) and natural or naturally derived oils (e.g., from plant materials or animal sources). In some embodiments, the oil comprises mineral oil. In some embodiments, the oil comprises a long chain fatty acid, a monoacylglycerol, a diacylglycerol, a triacylglycerol, or a combination thereof, wherein the acyl group is a long chain fatty acid. As used herein, “long chain fatty acid” refers to a carboxylic (CO₂H) acid having an aliphatic carbon chain of from about 11 to about 21 carbon atoms. The aliphatic carbon chain may be straight or branched. The aliphatic carbon chain may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the aliphatic carbon chain. Unsaturated alkyl groups may be mono- or polyunsaturated. Representative long chain fatty acids include, but are not limited to, undecylic acid, undecanoic acid, lauric acid, tridecanoic acid, myristic acid, pentadecanoic acid, palmitic acid, margaric acid, stearic acid, nonadecanoic acid, arachidic acid, heneicosanoic acid, α-linolenic acid, stearidonic acid, eicosapentaenoic acid, cervonic acid, linoleic acid, linolelaidic acid, γ-linolenic acid, dihomo-γ-linolenic acid, and arachidonic acid.

In some embodiments, the oil comprises an acyl glycerol, such as a monoacylglycerol, a diacylglycerol, or a triacylglycerol, wherein the acyl group is a long chain fatty acid as described herein. In some embodiments, the oil comprises a triacylglycerol, wherein the acyl group is a long chain fatty acid as described herein. In some embodiments, the oil comprises polyunsaturated long chain fatty acids, or mono-di- or triacylglycerol containing polyunsaturated long chain fatty acids as the acyl component. The chain lengths of the fatty acids in naturally occurring triglycerides may vary, but is typically 16, 18, or 20 carbon atoms. In some embodiments, the concentration of polyunsaturated fatty acid (as free fatty acid or as e.g., triglycerides) in the oil can range from about 2% to 100% (w/w), such as from about 5% to 100% (w/w) or greater than 10%, e.g., 20%-80% (w/w).

In some embodiments, the oil may be made up of primarily long-chain triacylglycerols (LCTs). In some embodiments, the oil may comprise medium-chain triacylglycerols (MCTs) and/or short-chain triacylglycerols (SCTs). In some embodiments, the oil comprises castor oil, corn oil, coconut oil, cod liver oil, evening primrose oil, cottonseed oil, palm oil, rice bran oil, sesame oil, rapeseed oil, canola oil, cocoa butter, linseed oil, olive oil, peanut oil, soybean oil, safflower oil, flaxseed oil, sunflower oil, olive oil, or a combination thereof.

The amount of oil present within the emulsion can vary. In some embodiments, the emulsion comprises oil in an amount of from about 1% to about 80% by weight, such as from about 5% to about 60% by weight, such as from about 5% to about 50% by weight, such as from about 5% to about 30% by weight, such as from about 10% to about 20% by weight, based on the total weight of the emulsion.

The emulsion may comprise water in the continuous or dispersed phase; i.e., the emulsion may comprise an aqueous phase. Water may be present as, for example, purified or ultrapure water, saline, buffered saline, or a buffered aqueous phase. The water present in the oral product may thus be at least partially contained in the emulsion. In some embodiments, the total amount of water present in the oral product is contained within the emulsion, such as within the aqueous phase of the emulsion. The water content of the emulsion may vary according to the desired properties. In some embodiments, the water content will be from about 10% to about 90% by weight, based on the total weight of the emulsion. In some embodiments, the water content is from about 15% to about 60% by weight, such as from about 20% to about 50% by weight, such as from about 25% to about 40% by weight, based on the total weight of the emulsion.

In some embodiments, a further hydrophilic, water soluble component may be added to the water, including short chain mono-, di-, and polyhydric alcohols, (e.g., ethanol, benzyl alcohol, glycerol, propylene glycol, propylene carbonate, polyethylene glycol with an average molecular weight of about 200 to about 10,000, diethylene glycol monoethyl ether, and combinations thereof).

Where present, the amount of the emulsion in the oral product may vary and may be any suitable amount for forming a product suitable for oral application. In some embodiments, the emulsion is present in the oral product in an amount of from about 1% to about 75% by weight of the oral product, such as from about 5% to about 60% by weight of the oral product, such as from about 10% to about 50% by weight of the oral product, such as from about 15% to about 45% by weight of the oral product, such as from about 20% to about 40% by weight of the oral product, such as from about 25% to about 40% by weight of the oral product, such as from about 30% to about 40% by weight of the oral product.

In some embodiments, the emulsion is present in the oral product in an amount of from about 20% to about 40% by weight of the oral product.

In some embodiments, the emulsion comprises a humectant. The humectant may be selected from the group consisting of glycerine, propylene glycol, 1,3-propanediol, dipropylene glycol, sorbitol, xylitol, mannitol, and mixtures thereof. In some embodiments, the emulsion comprises glycerine. The humectant may be present in the aqueous phase, the oil phase, or in both phases of the emulsion. In some embodiments, the humectant (such as glycerine) is present in the aqueous phase.

For the avoidance of doubt, combinations of the above end points are explicitly envisaged by the present disclosure. This applies to any of the ranges disclosed herein.

### Form of Emulsion

Where present, the emulsion may be in the form of a microemulsion.

In some embodiments, the emulsion is in the form of a nanoemulsion. A nanoemulsion is a colloidal particulate system with particulates in the submicron size range. The particulates (referred to herein also as droplets or particles) are generally solid spheres, and the surfaces of such particulates are amorphous and lipophilic with a negative charge. Nanoemulsions generally comprise nanoscale particles or droplets having an average size of less than about 1,000 nm. Nanoemulsions as described herein comprise nanoparticles (or nanodroplets) of the dispersed phase emulsified in the continuous phase. In some embodiments, the nanoemulsion comprises nanoparticles of an oil phase emulsified in water or the aqueous phase.

The nanoemulsion comprises the cannabinoid, preferably in the oil phase. Thus, in some embodiments, the oral product comprises a nanoemulsion comprising nanoparticles of an oil phase dispersed in an aqueous, wherein the cannabinoid is contained in the nanoparticles of the dispersed phase.

The nanoemulsion may further comprise an emulsifying agent. The relative amounts of these various components within the nanoemulsion may vary, and typically are selected so as to provide the desired sensory and performance characteristics to the nanoemulsion. Suitable amounts are described herein with reference generally to the emulsion.

Nanoemulsions as described herein generally comprise nanoscale particles having an average size of from about 10 nm to about 1,000 nm, for example, from about 10 nm to about 200 nm, from about 20 nm to about 100 nm, or from about 40 nm to about 100 nm. In some embodiments, the average particle size is about 100 nm, about 90 nm, about 80 nm, about 70 nm, about 60 nm, about 50 nm or about 40 nm. In some embodiments, the average particle size is from about 40 nm to about 80 nm. In some embodiments, the average particle size is from about 40 nm to about 80 nm, and the nanoemulsion is transparent.

The size of the nanoparticles may be determined by quasi-electric light scattering (QELS) as described in Bloomfield, Ann. Rev. Biophys. Bioeng., 10:421-450 (1981), incorporated herein by reference. It may also be measured by correlation spectroscopy that analyzes the fluctuation in scattering of light due to Brownian motion, or by transmission electron microscopy (TEM).

The nanoemulsion as described herein may be characterized by reference to a polydispersity index. Polydispersity indicates the uniformity of droplet size in a nanoemulsion. The higher the value of polydispersity, the lower will be the uniformity of droplet size. It may be defined as the ratio of standard deviation to mean droplet size. It may be measured by spectrophotometric methods. In some embodiments, it may be advantageous to provide nanoemulsions with a low polydispersity index, e.g., less than about 0.5. In some embodiments, the nanoemulsion has a polydispersity index of less than about 0.3.

Where present, the emulsion (such as the nanoemulsion) as described herein may be characterized by reference to zeta potential. Zeta potential is a measure of the charge on the surface of a droplet in the emulsion (or nanoemulsion). In some embodiments, the zeta potential of the nanoparticles is less than about −10 mV. In some embodiments, the zeta potential of the nanoparticles is less than about −20 mV. In some embodiments, the zeta potential of the nanoparticles is less than about −30 mV. In some embodiments, the zeta potential of the nanoparticles is less than about −40 mV. In some embodiments, the zeta potential of the nanoparticles is less than about −50 mV. In some embodiments, the zeta potential of the nanoparticles is from about −100 mV to about −10 mV, such as from about −100 mV to about −20 mV, such as from about −100 mV to about −30 mV, such as from about −100 mV to about −40 mV, such as from about −100 mV to about −50 mV. As appreciated by one skilled in the art, zeta potential is the measure of the electrical charge on particle surface in colloidal dispersions. Zeta potential may be measured with a zeta analyser, for example a Malvern Zetasizer.

In some embodiments, the weight ratio of the filler (such as microcrystalline cellulose) to the emulsion (such as a nanoemulsion) may be from about 10:1 to about 1:10, such as from about 5:1 to about 1:5, such as from about 5:1 to about 1:2, such as from about 3:1 to about 1:1, such as from about 2:1 to about 1:1.

### Emulsifying Agent

In embodiments in which the oral product comprises an emulsion, the oral product may further comprise one or more emulsifying agents. The one or more emulsifying agents may be contained within the emulsion. For example, the one or more emulsifying agents may be contained within the oil phase and/or the aqueous phase of an emulsion.

The emulsion (such as a nanoemulsion) in accordance with some embodiments may comprise one or more emulsifying agents. By “emulsifying agent” is meant a substance which aids in the formation and stabilization of emulsions by promoting dispersion of hydrophobic and hydrophilic (e.g., oil and water) components. In general, emulsifying agents are amphiphilic molecules chosen from, for example, nonionic and ionic amphiphilic molecules. The expression “amphiphilic molecule” means any molecule of bipolar structure comprising at least one hydrophobic portion and at least one hydrophilic portion and having the property of reducing the surface tension of water and of reducing the interface tension between water and an oily phase. Emulsifying agents/amphiphilic molecules as provided herein are also referred to as, for example, surfactants and emulsifiers.

The emulsifying agent may be included in the continuous phase, the dispersed phase, or in both the continuous phase and the dispersed phase of any emulsion. Alternatively or additionally, the emulsifying agent may be present at the interface of the dispersed and continuous phases.

In some embodiments, the emulsifying agent is selected from the group consisting of small molecule surfactants, phospholipids, proteins, polysaccharides, and mixtures thereof.

In some embodiments, the one or more emulsifying agents is selected from the group consisting of polyethylene glycol esters of fatty acids, propylene glycol esters of fatty acids, polysorbates, polyglycerol esters of fatty acids, polyglycerol polyricinoleate, sorbitan esters of fatty acid, sucrose esters of fatty acids, lecithins, enzyme treated lecithins, glycerin fatty acids esters, acetic acid esters of monoglycerides, lactic acid esters of monoglycerides, citric acid esters of monoglycerides, succinic acid esters of monoglycerides, diacetyl tartaric acid esters of monoglycerides, calcium stearoyl di lactate, chitin and chitosan derivatives, nature and modified starches, nature and modified hydrocolloids, nature and modified polysaccharides, nature and modified celluloses, nature and modified proteins, synthetic amphiphilic polymers, and mixtures thereof.

In some embodiments, the one or more emulsifying agents is selected from the group consisting of polyethylene glycol esters of fatty acids, propylene glycol esters of fatty acids, polysorbates, polyglycerol esters of fatty acids, polyglycerol polyricinoleate, sorbitan esters of fatty acid, sucrose esters of fatty acids, lecithins, glycerin fatty acids esters, acetic acid esters of monoglycerides, lactic acid esters of monoglycerides, citric acid esters of monoglycerides, succinic acid esters of monoglycerides, diacetyl tartaric acid esters of monoglycerides, calcium stearoyl di lactate, and mixtures thereof.

In some embodiments, the one or more emulsifying agents is selected from the group consisting of polyethylene glycol esters of fatty acids, polyethylene glycol esters of lecithin and mixtures thereof.

In some embodiments, the one or more emulsifying agents is selected from the group consisting of glycol distearate, sorbitan trioleate, sorbitan tristearate, sorbitan triisostearate, glyceryl isostearate, propylene glycol isostearate, glycol stearate, sorbitan sesquioleate, glyceryl stearate, lecithin, sorbitan oleate, sorbitan monostearate, sorbitan stearate, sorbitan isostearate, steareth-2, oleth-2, PEG-7 hydrogenated castor oil, laureth-2, sorbitan palmitate, laureth-3, glyceryl laurate, ceteth-2, PEG-30 dipolyhdroxystearate, glyceryl stearate SE, sorbitan stearate (and) sucrose cocoate, PEG-4 dilaurate, methyl glucose sesquistearate, PEG-8 dioleate, sorbitan laurate, PEG-40 sorbitan peroleate, laureth-4, PEG-7 glyceryl cocoate, PEG-20 almond glycerides, PEG-25 hydrogenated castor oil, stearamide MEA, glyceryl stearate (and) PEG-100 stearate, polysorbate 81, polysorbate 85, polysorbate 65, PEG-7 glyceryl cocoate, PEG-8 stearate, PEG-8 caprate, PEG-35 almond glycerides, PEG-6 laurate, laureth-7, steareth-10, isotrideceth-8, PEG-35 castor oil, isotrideceth-9, PEG-40 castor oil, ceteareth-12, laureth-9, PEG-40 hydrogenated castor oil, PEG-20 glyceryl isostearate, PEG-20 stearate, PEG-40 sorbitan perisostearate, PEG-7 olivate, cetearyl glucoside, PEG-8 oleate, polyglyceryl-3 methylglucose distearate, oleth-10, oleth-10/polyoxyl 10 oleyl ether NF, ceteth-10, PEG-8 laurate, cocamide MEA, polysorbate 60, polysorbate 80, isosteareth-20, PEG-60 almond glycerides, PEG-20 methyl glucose sesquistearate, ceteareth-20, oleth-20, steareth-20, steareth-21, ceteth-20, isoceth-20, polysorbate 20, polysorbate 40, ceteareth-25, ceteareth-30, PEG-30 stearate, laureth-23, PEG-75 lanolin, polysorbate 20, PEG-40 stearate, PEG-100 stearate, steareth-100, PEG-80 sorbitan laurate, polyoxyethylene stearate (e.g., polyoxyethylene (40) stearate), polyoxyethylene ether, and mixtures thereof.

In some embodiments, the one or more emulsifying agents have an overall HLB value in the range of from about 10 to about 15, such as from about 11 to about 15, such as from about 11 to about 14, such as from about 11 to about 13.5. As will be understood by one skilled in the art, HLB is the hydrophilic-lipophilic balance of an emulsifying agent or surfactant is a measure of the degree to which it is hydrophilic or lipophilic. The HLB value may be determined by calculating values for the different regions of the molecule, as described by Griffin in Griffin, William C. (1949), “Classification of Surface-Active Agents by ‘HLB’” (PDF), Journal of the Society of Cosmetic Chemists, 1 (5): 311-26 and Griffin, William C. (1954), “Calculation of HLB Values of Non-Ionic Surfactants” (PDF), Journal of the Society of Cosmetic Chemists, 5 (4): 249-56, and by Davies in Davies JT (1957), “A quantitative kinetic theory of emulsion type, I. Physical chemistry of the emulsifying agent” (PDF), Gas/Liquid and Liquid/Liquid Interface, Proceedings of the International Congress of Surface Activity, pp. 426-38. HLB value may be determined in accordance with the industry standard text book, namely “The HLB SYSTEM, a time-saving guide to emulsifier selection” ICI Americas Inc., Published 1976 and Revised, March, 1980. The HLB values of the emulsifiers described herein were determined in accordance with this standard method.

In some embodiments, the one or more emulsifying agents have an HLB value of from about 11 to about 15. In some embodiments, the one or more emulsifying agents have an HLB value of from about 11 to about 13.5. In some embodiments, the overall HLB value of the one or more emulsifying agents present in the oral product is from about 11 to about 15, such as from about 11 to about 13.5.

In some embodiments, the oral product comprises an emulsifying agent having an HLB value of from about 11 to about 15, wherein the emulsifying agent is selected from the group consisting of: stearamide MEA, glyceryl stearate (and) PEG-100 stearate, polysorbate 85, PEG-7 olivate, cetearyl glucoside, PEG-8 oleate, polyglyceryl-3 methylglucose distearate, oleth-10, oleth-10/polyoxyl 10 oleyl ether NF, ceteth-10, PEG-8 laurate, cocamide MEA, polysorbate 60, polysorbate 80, isosteareth-20, PEG-60 almond glycerides, PEG-20 methyl glucose sesquistearate, PEG-7 glyceryl cocoate, PEG-8 stearate, PEG-8 caprate, PEG-35 almond glycerides, PEG-6 laurate, laureth-7, steareth-10, isotrideceth-8, PEG-35 castor oil, isotrideceth-9, PEG-40 castor oil, ceteareth-12, laureth-9, PEG-40 hydrogenated castor oil, PEG-20 glyceryl isostearate, PEG-20 stearate, and mixtures thereof.

In some embodiments, the oral product comprises at least two emulsifying agents which have different HLB values. In some embodiments, the oral product comprises a first emulsifying agent with a low HLB value, and a second emulsifying agent with a high HLB value. In some embodiments, the oral product comprises a first emulsifying agent having an HLB value of from about 1 to about 9 (such as from about 2 to 9, such as from about 3 to 9, such as from about 3 to 8) and a second emulsifying agent having an HLB value of from about 10 to about 20 (such as from about 10 to 18, such as from about 11 to 17). In some embodiments, the overall (i.e., combined) HLB value of the first and second emulsifying agents is from about 11 to about 15, such as from about 11 to about 13.5.

The first emulsifying agent having an HLB value of from about 1 to about 9 may be selected from any suitable emulsifying agent having such an HLB value. For example, the first emulsifying agent may be an emulsifier having a HLB value of from about 1 to about 9 selected from mono and diglycerydes of fatty acid including glyceryl stearate and glyceryl oleate; fatty acid esters of C12-C22 fatty alcohols including fatty acid esters of cetyl alcohol and fatty acid esters of stearoyl alcohol, mixtures of fatty acid esters of cetyl alcohol and fatty acid esters of stearoyl alcohol, mixtures of fatty acid esters of cetyl alcohol and fatty acid esters of stearoyl alcohol wherein the fatty acids are derived from olive oil (such as cetearyl olivate), fatty acid esters of sorbitol including sorbitan oleate, fatty acid esters of sorbitol wherein the fatty acids are derived from olive oil (such as sorbitan olivate or cetearyl olivate), and mixtures thereof.

In some embodiments, the first emulsifying agent is an emulsifier having a HLB value of from about 1 to 9 selected from mono and diglycerydes of fatty acid, fatty acid esters of C12-C22 fatty alcohols, fatty acid esters of sorbitol, and mixtures thereof. In some embodiments, the first emulsifying agent is selected from the group consisting of glycol distearate, sorbitan trioleate, sorbitan tristearate, sorbitan triisostearate, glyceryl isostearate, propylene glycol isostearate, glycol stearate, sorbitan sesquioleate, glyceryl stearate, lecithin (such as soy lecithin), sorbitan oleate, sorbitan monostearate, sorbitan stearate, sorbitan isostearate, steareth-2, oleth-2, PEG-7 hydrogenated castor oil, laureth-2, sorbitan palmitate, laureth-3, glyceryl laurate, ceteth-2, PEG-30 dipolyhdroxystearate, glyceryl stearate SE, sorbitan stearate (and) sucrose cocoate, PEG-4 dilaurate, methyl glucose sesquistearate, PEG-8 dioleate, sorbitan laurate, PEG-40 sorbitan peroleate, and mixtures thereof.

In some embodiments, the first emulsifying agent is or comprises lecithin. In some embodiments, the first emulsifying agent is or comprises soy lecithin.

The second emulsifying agent may be selected from any suitable emulsifying agent having an HLB value of from about 10 to about 20. In some embodiments, the second emulsifying agent is an emulsifier having a HLB value of from 10 to 20 selected from fatty acid esters of polyethylene glycol, such as fatty acid esters of polyethylene glycol wherein the fatty acids are derived from coconut oil (including PEG 7), fatty acid esters of polyglycerol including fatty acid esters of polyglycerol and oleic acid (such as polyglyceryl 10 oleate), and mixtures thereof. In some embodiments, the second emulsifying agent is an emulsifier having a HLB value of from 10 to 20 selected from fatty acid esters of polyethylene glycol, fatty acid esters of polyglycerol, and mixtures thereof. In some embodiments, the second emulsifying agent may be selected from the group consisting of laureth-4, PEG-7 glyceryl cocoate, PEG-20 almond glycerides, PEG-25 hydrogenated castor oil, stearamide MEA, glyceryl stearate (and) PEG-100 stearate, polysorbate 81, polysorbate 85, polysorbate 65, PEG-7 glyceryl cocoate, PEG-8 stearate, PEG-8 caprate, PEG-35 almond glycerides, PEG-6 laurate, laureth-7, steareth-10, isotrideceth-8, PEG-35 castor oil, isotrideceth-9, PEG-40 castor oil, ceteareth-12, laureth-9, PEG-40 hydrogenated castor oil, PEG-20 glyceryl isostearate, PEG-20 stearate, PEG-40 sorbitan perisostearate, PEG-7 olivate, cetearyl glucoside, PEG-8 oleate, polyglyceryl-3 methylglucose distearate, oleth-10, oleth-10/polyoxyl 10 oleyl ether NF, ceteth-10, PEG-8 laurate, cocamide MEA, polysorbate 60, polysorbate 80, isosteareth-20, PEG-60 almond glycerides, PEG-20 methyl glucose sesquistearate, ceteareth-20, oleth-20, steareth-20, steareth-21, ceteth-20, isoceth-20, polysorbate 20, polysorbate 40, ceteareth-25, ceteareth-30, PEG-30 stearate, laureth-23, PEG-75 lanolin, polysorbate 20, PEG-40 stearate, PEG-100 stearate, steareth-100, PEG-80 sorbitan laurate, polyoxyethylene stearate (e.g., polyoxyethylene (40) stearate), polyoxyethylene ether, and mixtures thereof.

In some embodiments, the second emulsifying agent is or comprises polyoxyethylene stearate (e.g., polyoxyethylene (40) stearate).

In some embodiments, the emulsifying agent is or comprises a combination of lecithin (e.g., soy lecithin) and polyoxyethylene stearate (e.g., polyoxyethylene (40) stearate).

In some embodiments, the one or more emulsifying agents comprises neutral, positively charged, or negatively charged natural or synthetic phospholipids molecules. Phospholipids are made up of two fatty acid tails and a phosphate group head, connected via a third molecule, glycerol. Non-limiting examples of natural phospholipids including lecithin (such as soy lecithin and/or egg lecithin), phosphatidyl choline-enriched lecithin, phosphatidyl serine-enriched lecithin, enzymatically modified lecithin, phosphatidylglycerol, phosphatidylinositol, phosphatidylethanolamine, phosphatidic acid, sphingomyelin, diphosphatidylglycerol, phosphatidylserine, phosphatidylcholine and cardiolipin; synthetic phospholipids including dimyristoylphosphatidylcholine, dimyristoylphosphatidylglycerol, di stearoylphosphatidylglycerol and dipalmitoylphosphatidylcholine; and hydrogenated or partially hydrogenated lecithins and phospholipids. Non-limiting examples of synthetic phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), PEG phospholipid (mPEG-phospholipid, polyglycerin-phospholipid, functionalized-phospholipid, and terminal activated-phospholipid).

In some embodiments, the emulsifying agent comprises a surfactant, which may be ionic (anionic or cationic), zwitterionic or non-ionic, and which may be hydrophobic or hydrophilic. Examples of hydrophobic surfactants include, but are not limited to, Maisine 35-1, Imwitor 742, Capmul MCM, Capmul PG 12, Lauroglycol 90, Lauroglycol FCC, Caproyl 90, Captex 250, a fatty acid selected from the group consisting of octanoic acid, decanoic acid, undecanoic acid, lauric acid, myristic acid, palmitic acid, stearic acid, oleic acid, linoleic acid, and linolenic acid. As used herein, a hydrophobic surfactant may also be referred to as a poorly water soluble surfactant or a lipophilic surfactant.

Examples of hydrophilic surfactants may include, but are not limited to polyoxyethylene sorbitan fatty acid esters, hydrogenated castor oil ethoxylates, PEG mono- and di-esters of palmitic and stearic acids, fatty acid ethoxylates, and combinations thereof.

Examples of suitable surfactants generally include, but are not limited to: polyoxyethylene-sorbitan-fatty acid esters; e.g., mono- and tri-lauryl, palmityl, stearyl and oleyl esters; e.g., products of the type known as polysorbates and commercially available under the trade name Tween®; polyoxyethylene fatty acid esters, e.g., polyoxyethylene stearic acid esters of the type known and commercially available under the trade name Myrj (ID; polyoxyethylene ethers, such as those available under the trade name Brij®; polyoxyethylene castor oil derivatives, e.g., products of the type known and commercially available as Cremophors®. Particularly suitable are polyoxyl 35 castor oil (Cremophor®EL) and polyoxyl 40 hydrogenated castor oil (Cremophor®RH40); a-tocopherol, a-tocopheryl polyethylene glycol succinate (vitamin E TPGS), a-tocopherol palmitate and a-tocopherol acetate; PEG glyceryl fatty acid esters such as PEG-8 glyceryl caprylate/caprate (commercially known as Labrasol®), PEG-4 glyceryl caprylate/caprate (Labrafac Hydro WL 1219), PEG-32 glyceryl laurate (Gelucire 44/14), PEG-6 glyceryl mono oleate (Labrafil® M 1944 CS), PEG-6 glyceryl linoleate (Labrafil® M 2125 CS); propylene glycol mono- and di-fatty acid esters, such as propylene glycol laurate, propylene glycol caprylate/caprate; also diethyleneglycol-monoethylether (DGME), commercially known as Transcutol® (Gattefosse, Westwood, N.J.); sorbitan fatty acid esters, such as the type known and commercially available under the name Span® (e.g., Span 85); polyoxyethylene-polyoxypropylene co-polymers, e.g., products of the type known and commercially available as Pluronic® or Poloxamer®; glycerol triacetate; and monoglycerides and acetylated monoglycerides, e.g., glycerol monodicocoate (Imwitor® 928), glycerol monocaprylate (Imwitor® 308), and mono- and di-acetylated monoglycerides.

In some embodiments, the emulsifying agent is a surfactant, a phospholipid, an amphiphilic polysaccharide, an amphiphilic protein, or a combination thereof. In some embodiments, the one or more emulsifying agents is an ionic, zwitterionic, or non-ionic surfactant. In some embodiments, the one or more emulsifying agents comprises Tween 20, Tween 80, Span 20, Span 40, Span 60, Span 80, lecithin, Myrj 52, Brij 35, Brij 97, a hydrocolloid gum, a modified starch, or a combination thereof. In some embodiments, the one or more emulsifying agents comprises a combination of lecithin and Myrj 52.

The concentration of the one or more emulsifying agents present in the disclosed emulsion may vary. The total concentration of the emulsifying agent may be in a range of up to about 30% by weight, for example from about 0.1% to about 25%, from about 5% to about 25%, or from about 10% to about 25% by weight based on the entirety of the emulsion. In some embodiments, the emulsion comprises a combination of lecithin and Myrj 52 in an amount of from about 0.1% to about 25%, from about 5% to about 25%, or from about 10% to about 25% by weight based on the entirety of the emulsion. In some embodiments, the one or more emulsifying agents may be present in the emulsion in an amount of from about 0.1% to about 20% by weight of the oral product, such as from about 1% to about 15% by weight of the oral product, such as from about 2.5% to about 10% by weight of the oral product, such as from about 5% to about 10% by weight of the oral product. In some embodiments, the emulsion comprises a combination of lecithin and Myrj 52 in an amount of from about 0.1% to about 20% by weight of the oral product, such as from about 1% to about 15% by weight of the oral product, such as from about 2.5% to about 10% by weight of the oral product, such as from about 5% to about 10% by weight of the oral product.

### Stabilizer

In embodiments in which the oral product comprises an emulsion, the oral product may further comprise a stabilizing agent (or “stabilizer”). The stabilizer may assist in maintaining the (nano)emulsion. Representative examples of suitable types of stabilizers include polysaccharides, polyols, sorbitan esters, glycerol esters, polyethylene glycol esters, block polymers, acrylic polymers (such as Pemulen), silicon based surfactants, and polysorbates. In some embodiments, the stabilizer is sodium oleate, glycerine, xylitol, sorbitol, ascorbic acid, sodium edetate, a sorbitan ester, a glycerol monoester, or a combination thereof.

The concentration of the stabilizer present in the emulsion may vary. When present, the concentration of the stabilizer may be in a range of up to about 10% by weight, for example from about 0.01% to about 10%, from about 0.1% to about 5%, or from about 0.5% to about 1% by weight based on the weight of the emulsion.

### Total Product

In some embodiments, the oral product comprises:
(i) a cannabinoid;
(ii) a cellulose material selected from the group consisting of sugar beet fiber, wood pulp fiber, bamboo fiber, derivatives thereof, and combinations thereof; and
(iii) water;

wherein the water content of the oral product is at least about 10% by weight of the oral product;

and wherein the water activity of the oral product is no greater than about 0.85.

In some embodiments, the oral product comprises:
(i) cannabidiol;
(ii) a cellulose material selected from the group consisting of sugar beet fiber, wood pulp fiber, bamboo fiber, derivatives thereof, and combinations thereof; and
(iii) water;

wherein the water content of the oral product is at least about 10% by weight of the oral product;

and wherein the water activity of the oral product is no greater than about 0.85.

In some embodiments, the oral product comprises:
(i) a cannabinoid;
(ii) microcrystalline cellulose; and
(iii) water;

wherein the water content of the oral product is at least about 10% by weight of the oral product;

and wherein the water activity of the oral product is no greater than about 0.85.

In some embodiments, the oral product comprises:
(i) cannabidiol;
(ii) microcrystalline cellulose; and
(iii) water;

wherein the water content of the oral product is at least about 10% by weight of the oral product;

and wherein the water activity of the oral product is no greater than about 0.85.

In some embodiments, the oral product comprises:
(i) a cannabinoid;
(ii) a cellulose material selected from the group consisting of sugar beet fiber, wood pulp fiber, bamboo fiber, derivatives thereof, and combinations thereof; and
(iii) water;

wherein the water content of the oral product is from about 10% to about 30% by weight of the oral product;

and wherein the water activity of the oral product is no greater than about 0.8.

In some embodiments, the oral product comprises:
(i) a cannabinoid;
(ii) a cellulose material selected from the group consisting of sugar beet fiber, wood pulp fiber, bamboo fiber, derivatives thereof, and combinations thereof;
(iii) water; and
(iv) a humectant;

wherein the water content of the oral product is at least about 10% by weight of the oral product;

and wherein the water activity of the oral product is no greater than about 0.85.

In some embodiments, the oral product comprises:
(i) a cannabinoid;
(ii) a cellulose material selected from the group consisting of sugar beet fiber, wood pulp fiber, bamboo fiber, derivatives thereof, and combinations thereof;
(iii) water; and
(iv) a humectant selected from the group consisting of glycerine, propylene glycol and mixtures thereof;

wherein the water content of the oral product is at least about 10% by weight of the oral product;

and wherein the water activity of the oral product is no greater than about 0.85.

In some embodiments, the oral product comprises:
(i) a cannabinoid;
(ii) a cellulose material selected from the group consisting of sugar beet fiber, wood pulp fiber, bamboo fiber, derivatives thereof, and combinations thereof;
(iii) water; and
(iv) a salt;

wherein the water content of the oral product is at least about 10% by weight of the oral product;

and wherein the water activity of the oral product is no greater than about 0.85.

In some embodiments, the oral product is configured such that the water activity is no greater than about 0.85, such as no greater than about 0.8, such as no greater than about 0.75, such as no greater than about 0.7, such as no greater than about 0.6, such as no greater than about 0.5. It was found by the present inventors that, when the water activity of the oral product was reduced to below 0.85, the oral product could be stored for a period of several weeks or months without exhibiting significant microbiological growth.

As described herein, the “water activity” (a_{w}) of the oral product is the partial vapor pressure of the water in the product divided by the standard state partial vapor pressure of water. Water activity may be calculated using the following formula:

$${a}_{w} = \frac{ρ}{{ρ}^{*}}$$

where ρ is the partial vapor pressure of water in the product, and ρ* is the partial vapor pressure of pure water at the same temperature. The water activity may be measured using any known and suitable measurement method in the art. In some embodiments, the water activity is measured using a resistive electrolytic hygrometer. In some embodiments, the water activity is measured using a capacitance hygrometer. In some embodiments, the water activity is measured using a dew point hygrometer. In some embodiments, the water activity is measured using a water activity meter having a tuneable diode laser.

In some embodiments, the water activity of the oral product is from about 0.1 to about 0.8, such as from about 0.5 to about 0.8, such as from about 0.6 to about 0.8, such as from about 0.7 to about 0.8, such as from about 0.73 to about 0.78.

In particular, it has been surprisingly found by the present inventors that, when a cannabinoid is included in an oral product in combination with a filler and water in an amount of at least 10% by weight as described herein and in which the water activity of the oral product is less than about 0.85, a product having an improved mouthfeel and an improved chemical/physical stability and shelf-life may be provided.

It is desirable for the product to have a shelf-life such that it can be stored for a period of several days, weeks or months.

It was also found that the oral product may be both chemically and physically stable for a period of at least 6 months, for example at a relative humidity of 50%. By “chemically and physically stable”, it is understood that the cannabinoid does not migrate out of the product as such a migration will lead to a marked loss of the cannabinoid in the product (chemical stability), and also that no visible changes are observed over the measured period (physical stability) and the dissolution profile does not change.

It was found in particular that the water activity of the product could be reduced via a number of different means. For example, one or more preservatives could be included. Alternatively or in addition, a humectant and/or salt may be included to reduce the amount of free water in the oral product. As described herein, in some embodiments, the oral product thus comprises a humectant and/or a salt in an amount suitable for reducing the water activity to no greater than 0.85. Alternatively or in addition, the amount of the filler (such as a cellulose material, such as microcrystalline cellulose) may be increased to an amount of at least about 50% by weight such that the water activity is reduced. In some embodiments, the oral product may comprise water in an amount of less than about 30% by weight of the oral product, such as less than about 25% by weight of the oral product, such as less than about 20% by weight of the oral product, such as less than about 15% by weight of the oral product.

It has also been surprisingly found that, when a filler as defined herein is combined with a cannabinoid in an oral product having a water activity of no greater than about 0.85, the release of the cannabinoid into the user's mouth may be relatively rapid. In some embodiments, when placed in the oral cavity of a user, the oral product releases at least 50% by weight of the cannabinoid within at the most about 60 minutes, such as at the most about 45 minutes, such as at the most about 30 minutes, such as at the most about 15 minutes, such as at the most about 10 minutes, such as at the most about 5 minutes. In some embodiments, when placed in the oral cavity of a user, the oral product releases at least 60% by weight of the cannabinoid within at the most about 60 minutes, such as at the most about 45 minutes, such as at the most about 30 minutes, such as at the most about 15 minutes, such as at the most about 10 minutes, such as at the most about 5 minutes. In some embodiments, when placed in the oral cavity of a user, the oral product releases at least 70% by weight of the cannabinoid within at the most about 60 minutes, such as at the most about 45 minutes, such as at the most about 30 minutes, such as at the most about 15 minutes, such as at the most about 10 minutes, such as at the most about 5 minutes. In some embodiments, when placed in the oral cavity of a user, the oral product releases at least 80% by weight of the cannabinoid within at the most about 60 minutes, such as at the most about 45 minutes, such as at the most about 30 minutes, such as at the most about 15 minutes, such as at the most about 10 minutes, such as at the most about 5 minutes. In some embodiments, when placed in the oral cavity of a user, the oral product releases at least 90% by weight of the cannabinoid within at the most about 60 minutes, such as at the most about 45 minutes, such as at the most about 30 minutes, such as at the most about 15 minutes, such as at the most about 10 minutes, such as at the most about 5 minutes. In some embodiments, when placed in the oral cavity of a user, the oral product releases at least 95% by weight of the cannabinoid within at the most about 60 minutes, such as at the most about 45 minutes, such as at the most about 30 minutes, such as at the most about 15 minutes, such as at the most about 10 minutes, such as at the most about 5 minutes.

The rate of release into the oral cavity may be measured using an in vitro dissolution test. The dissolution profile of the cannabinoid may be measured as the amount of cannabinoid released after a certain period of time in 1 litre of phosphate buffer and at a pH of about 7.4 maintained at 37° C. using a USP paddle dissolution apparatus.

It has also been found that, by combining a filler as defined herein with a cannabinoid in an oral product in which the water activity is no greater than about 0.85, it may be possible to incorporate the cannabinoid in the form of an emulsion that contains water in an aqueous phase whilst maintaining an acceptable shelf-life. When the cannabinoid is contained in the form of an emulsion, the release characteristics and rate of absorption of the cannabinoid into the oral mucosa may be further improved. Indeed, it has been found by the inventors that, by including the cannabinoid in an emulsion, the problems associated with lack of water solubility are overcome. The cannabinoid may be released from the oral product and into the mouth of the user within a relatively short period of time. Furthermore, the cannabinoid is readily absorbed into the oral mucosa, and thus into the bloodstream, without the need for swallowing the active agent. The physiological effects of the active are therefore felt much more rapidly by the user than with previously known formulations.

In some embodiments, at least 30% by weight of the released cannabinoid (i.e., that which has been released into the oral cavity of the user over the period of time specified) is absorbed into the oral mucosa within at the most about 60 minutes, such as at the most about 45 minutes, such at the most about 30 minutes, such as at the most about 15 minutes, such as at the most about 10 minutes, such as at the most about 5 minutes. In some embodiments, at least 40% by weight of the released cannabinoid (i.e., that which has been released into the oral cavity of the user over the period of time specified) is absorbed into the oral mucosa within at the most about 60 minutes, such as at the most about 45 minutes, such as at the most about 30 minutes, such as at the most about 15 minutes, such as at the most about 10 minutes, such as at the most about 5 minutes. In some embodiments, at least 50% by weight of the released cannabinoid (i.e., that which has been released into the oral cavity of the user over the period of time specified) is absorbed into the oral mucosa within at the most about 60 minutes, such as at the most about 45 minutes, such as at the most about 30 minutes, such as at the most about 15 minutes, such as at the most about 10 minutes, such as at the most about 5 minutes. In some embodiments, at least 60% by weight of the released cannabinoid (i.e., that which has been released into the oral cavity of the user over the period of time specified) is absorbed into the oral mucosa within at the most about 60 minutes, such as at the most about 45 minutes, such as at the most about 30 minutes, such as at the most about 15 minutes, such as at the most about 10 minutes, such as at the most about 5 minutes. In some embodiments, at least 70% by weight of the released cannabinoid (i.e., that which has been released into the oral cavity of the user over the period of time specified) is absorbed into the oral mucosa within at the most about 60 minutes, such as at the most about 45 minutes, such as at the most about 30 minutes, such as at the most about 15 minutes, such as at the most about 10 minutes, such as at the most about 5 minutes. In some embodiments, at least 75% by weight of the released cannabinoid (i.e., that which has been released into the oral cavity of the user over the period of time specified) is absorbed into the oral mucosa within at the most about 60 minutes, such as at the most about 45 minutes, such as at the most about 30 minutes, such as at the most about 15 minutes, such as at the most about 10 minutes, such as at the most about 5 minutes.

In some embodiments, the oral product releases the cannabinoid such that at least about 20% by weight of the cannabinoid is absorbed into the oral mucosa (e.g., gingival or buccal mucosa) of the user within at the most about 60 minutes, such as at the most about 45 minutes, such as at the most about 30 minutes, such as at the most about 15 minutes, such as at the most about 10 minutes, such as at the most about 5 minutes. In some embodiments, the oral product releases the cannabinoid such that at least about 25% by weight of the cannabinoid is absorbed into the oral mucosa of the user within at the most about 60 minutes, such as at the most about 45 minutes, such as at the most about 30 minutes, such as at the most about 15 minutes, such as at the most about 10 minutes, such as at the most about 5 minutes. In some embodiments, the oral product releases the cannabinoid such that at least about 30% by weight of the cannabinoid is absorbed into the oral mucosa of the user within at the most about 60 minutes, such as at the most about 45 minutes, such as at the most about 30 minutes, such as at the most about 15 minutes, such as at the most about 10 minutes, such as at the most about 5 minutes. In some embodiments, the oral product releases the cannabinoid such that at least about 40% by weight of the cannabinoid is absorbed into the oral mucosa of the user within at the most about 60 minutes, such as at the most about 45 minutes, such as at the most about 30 minutes, such as at the most about 15 minutes, such as at the most about 10 minutes, such as at the most about 5 minutes. In some embodiments, the oral product releases the cannabinoid such that at least about 50% by weight of the cannabinoid is absorbed into the oral mucosa of the user within at the most about 60 minutes, such as at the most about 45 minutes, such as at the most about 30 minutes, such as at the most about 15 minutes, such as at the most about 10 minutes, such as at the most about 5 minutes.

The percentage amount of absorption may be measured in vitro. For example, the extent of absorption of the cannabinoid into the oral mucosa may be measured via octanol-water partitioning. For example, the product may be dissolved in saliva at about 37° C., and then extracted using octanol as part of a liquid-liquid extraction step. The percentage amount of active ingredient absorbed into the oral mucosa (i.e., degree of in vitro absorption) thus corresponds to the percentage amount that is extracted into the octanol.

Release characteristics and rates of absorption of the cannabinoid into the oral mucosa may be measured by any suitable means. For example, techniques known to one skilled in the art for the measurement of release and absorption of nicotine may be used.

### Process

In accordance with some embodiments described herein, there is provided a process for preparing an oral product as defined herein, the process comprising:
(a) providing a cannabinoid, a filler and water, and
(b) contacting the cannabinoid, the filler and the water to form an oral product;
   wherein the water activity of the oral product is no greater than about 0.85.

In some embodiments, the process further comprises contacting one or more additives with the filler and/or the cannabinoid. Such additives may be added before, during and/or after (b). In some embodiments, one or more additives is contacted with the filler or cannabinoid before (b). In some embodiments, one or more additives is contacted with the filler and cannabinoid during (b). In some embodiments, one or more additives is contacted with the filler and cannabinoid after (b).

The oral product, filler and cannabinoid, and optionally one or more additives, may be as described hereinabove.

The manner by which the various components of the composition are combined may vary. As such, the overall composition with e.g., powdered composition components may be relatively uniform in nature. The components noted above, which may be in liquid or dry solid form, can be admixed in a pretreatment prior to mixture with any remaining components of the composition, or simply mixed together with all other liquid or dry ingredients. The various components of the composition may be contacted, combined, or mixed together using any mixing technique or equipment known in the art. Any mixing method that brings the composition ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference. In some embodiments, the components forming the composition are prepared such that the mixture thereof may be used in a molding process for forming the composition. Manners and methods for formulating compositions will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and U.S. Pat. No. 6,077,524 to Bolder et al., each of which is incorporated herein by reference.

In some embodiments, any one or more component, and the overall oral product described herein, can be described as a particulate material. As used herein, the term “particulate” refers to a material in the form of a plurality of individual particles, some of which can be in the form of an agglomerate of multiple particles, wherein the particles have an average length to width ratio less than 2:1, such as less than 1.5:1, such as about 1:1. In various embodiments, the particles of a particulate material can be described as substantially spherical or granular.

The particle size of a particulate material may be measured by sieve analysis. As the skilled person will readily appreciate, sieve analysis (otherwise known as a gradation test) is a method used to measure the particle size distribution of a particulate material. Typically, sieve analysis involves a nested column of sieves which comprise screens, preferably in the form of wire mesh cloths. A pre-weighed sample may be introduced into the top or uppermost sieve in the column, which has the largest screen openings or mesh size (i.e., the largest pore diameter of the sieve). Each lower sieve in the column has progressively smaller screen openings or mesh sizes than the sieve above. Typically, at the base of the column of sieves is a receiver portion to collect any particles having a particle size smaller than the screen opening size or mesh size of the bottom or lowermost sieve in the column (which has the smallest screen opening or mesh size).

In some embodiments, the column of sieves may be placed on or in a mechanical agitator. The agitator causes the vibration of each of the sieves in the column. The mechanical agitator may be activated for a pre-determined period of time in order to ensure that all particles are collected in the correct sieve. In some embodiments, the column of sieves is agitated for a period of time from 0.5 minutes to 10 minutes, such as from 1 minute to 10 minutes, such as from 1 minute to 5 minutes, such as for approximately 3 minutes. Once the agitation of the sieves in the column is complete, the material collected on each sieve is weighed. The weight of each sample on each sieve may then be divided by the total weight in order to obtain a percentage of the mass retained on each sieve. As the skilled person will readily appreciate, the screen opening sizes or mesh sizes for each sieve in the column used for sieve analysis may be selected based on the granularity or known maximum/minimum particle sizes of the sample to be analysed. In some embodiments, a column of sieves may be used for sieve analysis, wherein the column comprises from 2 to 20 sieves, such as from 5 to 15 sieves. In some embodiments, a column of sieves may be used for sieve analysis, wherein the column comprises 10 sieves. In some embodiments, the largest screen opening or mesh sizes of the sieves used for sieve analysis may be 1000 μm, such as 500 μm, such as 400 μm, such as 300 μm.

In some embodiments, any material referenced herein (e.g., filler, and the overall oral product) characterized as being in particulate form may have at least 50% by weight of particles with a particle size as measured by sieve analysis of no greater than about 1000 μm, such as no greater than about 500 μm, such as no greater than about 400 μm, such as no greater than about 350 μm, such as no greater than about 300 μm. In some embodiments, at least 60% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 70% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 80% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 90% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 95% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 99% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, approximately 100% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm.

In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 0.01 μm to about 1000 μm, such as from about 0.05 μm to about 750 μm, such as from about 0.1 μm to about 500 μm, such as from about 0.25 μm to about 500 μm. In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 10 μm to about 400 μm, such as from about 50 μm to about 350 μm, such as from about 100 μm to about 350 μm, such as from about 200 μm to about 300 μm.

In some embodiments, the method comprises mixing the filler, at least one cannabinoid, and a salt to form a first mixture; and adding water to the first mixture to form the final oral product. In some embodiments, the method further comprises adding one or more binders to the first mixture. In some embodiments, the method further comprises adding a buffer, one or more sweeteners, a humectant, a flavoring agent, a taste modifying agent, or a combination thereof, to the first mixture. In some embodiments, the method further comprises adding additional water to the composition in order to provide the final oral product.

In another aspect is provided an oral product obtained or obtainable by the method as disclosed herein. In another aspect is provided an oral product prepared by the method as disclosed herein.

In some embodiments, the oral product comprises an emulsion. The emulsion may be as described hereinabove, and may include any of the features or combinations of features as described herein.

In some embodiments, providing a cannabinoid and/or water may comprise forming the emulsion containing the cannabinoid and/or water. Feature (a) of providing the cannabinoid and/or water may comprise mixing an oil phase with an aqueous phase, optionally in the presence of an emulsifying agent, so as to form the emulsion.

In some embodiments, the emulsion is a nanoemulsion. Nanoemulsions may be prepared using a high-energy method or a low-energy method. High-energy methods utilize mechanical devices (homogenizers) that are capable of generating intense disruptive forces that are capable of disrupting the oil and aqueous phases into tiny oil droplets (see McClements and Rao, Critical Reviews in Food Science and Nutrition, 51, 285-330 (2011)). Such high-energy approaches include the use of high pressure valve homogenizers, microfluidizers, and sonication methods. Low-energy approaches may rely on the spontaneous formation of tiny oil droplets within systems when the solution or environmental conditions are altered.

For example, nanoemulsions as disclosed herein can be prepared by mechanical processes which employ shear force to break large emulsion droplets into smaller ones, such as high-pressure homogenization (HPH, including microfluidization), high-amplitude ultrasonic processing, and ultrasound-assisted emulsification. In some embodiments, the nanoemulsion may be formed via the use of a high pressure valve homogenizer, a microfluidizer, or an ultrasonic homogenizer (including ultrasonic jet homogenizers and ultrasonic probe homogenizers). In general, the nanoemulsions of the present disclosure can be prepared by preparing an aqueous phase containing an emulsifying agent as disclosed herein (e.g., an amphiphilic molecule or surfactant) and homogenizing this solution with a homogenizer or mixer for a period of time; and preparing an oil phase containing an oil, as described herein above.

At least one cannabinoid may be added to the aqueous and/or oil phase; in addition, one or more further hydrophobic active ingredients, flavors, or combinations thereof, as desired, may be added to the aqueous and/or oil phase. Such addition may be followed by mixing the same with a suitable mixing device. The aqueous and oil phases are combined and homogenized with, for example, a probe sonicator (Sonics and Materials, USA), a high pressure homogenizer (such as one made by Gauline or Avestine, or the like), or a microfluidizer, to obtain the desired nanoemulsion. The number of passes through a high pressure homogenizer/microfluidizer may vary, depending on the desired particle size for the nanoemulsions. A variety of methods are known in the art for producing nanoemulsions comprising nano-sized particles of particular size ranges, using for example, sonication or homogenization. One such method is described in U.S. Pat. No. 4,737,323, incorporated herein by reference.

In some embodiments, (b) of the above-described method comprises combining the emulsion (such as a nanoemulsion) as described above with the filler. For example, the filler may be microcrystalline cellulose. In some embodiments, the filler may be present in an amount of at least 50% by weight of the oral product.

In some embodiments in which the cannabinoid is provided in the form of an emulsion, the process further comprises (a)(i) of combining a filler (such as a cellulose material, such as microcrystalline cellulose) with a salt, sweetener and/or flavoring agent. The emulsion may then be combined with the resultant product from (a)(i) during (b) in order to form the oral product.

### EXAMPLES

Aspects of the present invention are more fully illustrated by the following examples, which are set forth to illustrate certain aspects of the present invention and are not to be construed as limiting thereof.

### Example 1—Oral Product

Samples of oral products according to embodiments of the present disclosure are prepared from a composition comprising cannabidiol as the cannabinoid, and microcrystalline cellulose (MCC), water, and additional components as disclosed herein (salt, sweeteners, humectant, buffer, and flavoring agent).

The oral product is prepared by combining microcrystalline cellulose (57% of the composition by weight), cannabidiol (6% of the composition by weight), a salt (4% of the composition by weight) and a sweetener—acesulfame K—(0.7% of the composition by weight) to form a mixture of dry ingredients. To the mixture of dry ingredients was added water (13.3% of the composition by weight), buffer—sodium bicarbonate (1% of the composition by weight), glycerine (15% of the composition by weight), sodium benzoate (1% of the composition by weight) and flavoring agent (2% of the composition by weight).

The water activity of the composition is found to be no greater than 0.85.

### Example 2—Pouched Oral Product

Portions of the oral product of Example 1 (442.4 mg) are placed into pouches for a product weight of 476 mg. The pouches are composed of a fleece material with polyester fibers and an acrylic binder. After incorporation of the oral product into the pouches, the pouch material is heat-sealed in order to provide the pouched product.

### Example 3—Oral Product Comprising Emulsion

### Preparation of Emulsion

A process according to embodiments of the present disclosure is utilized in order to prepare an emulsion comprising an oil phase and a water phase, and a cannabinoid as the active ingredient.

The emulsion is prepared by mixing castor oil with an isolate of cannabidiol in a weight ratio of 3:1 to prepare the oil phase. The mixture is heated at about 70° C. for a period of about 10 minutes until the mixture has become transparent.

The aqueous phase is formed by mixing water with a preservative (sodium benzoate) and an emulsifying agent (combination of Myrj 52 and lecithin). The amount of preservative included is 0.4% by weight of the aqueous phase, and the amount of emulsifying agent is 20% by weight of the aqueous phase. Glycerine is also added to the water in an amount of 35% by weight of the aqueous phase. The components of the aqueous phase are subjected to high shear mixing for a period of 20 minutes. High shearing mixer is used to an initial emulsion prior to ultrasonic homogenization step. An Ika Ultra-turrax disperser is utilized to prepare homogenous slurry of solid ingredients in water and to fabricate the initial emulsion thereafter. Typically, 5000-15000 rpm shear rate is needed for prepare aqueous slurry and initial emulsion.

The oil phase and aqueous phase are then combined in a weight ratio of 1:9 to provide mixture having the following components:

| | | Amount (% w/w |
|---|---|---|
| Raw Material | | of emulsion) |
| Oil Phase | Castor Oil | 7.5 |
| | Cannabidiol | 2.5 |
| Aqueous Phase | Water | 40.14 |
| | Myrj 52 | 9 |
| | Lecithin | 9 |
| | Glycerine | 31.5 |
| | Sodium | 0.36 |
| | benzoate | |

The oil phase and aqueous phases are combined via high shear mixing for a period of about 20 minutes at 30° C. or until a homogenous opaque emulsion forms.

The resulting macroemulsion is then added to an ultrasonic probe homogenizer (i.e., sonicator) feed vat and the temperature set to 30° C. The macroemulsion is flowed through the sonicator at 150 mL/min, and using instrument specific amplitude of 80 μm. The temperature leaving the sonicator does not exceed 40° C. A Fisherbrand model 505 ultrasonic homogenizer with max. 500 watt output is used for the present batch preparation. A Hielscher UIP4000hdT ultrasonic homogenizer is used for larger scale batch production. Typical operating parameters of Hielscher ultrasonic homogenizer are 15 liter/hour (flow rate), 21 to 66° C. (temperature range) and 7 hours (operation time per day). Parameters may be adjusted during productions to optimize the output and quality of the products. The resulting nanoemulsion is then passed through a filter (1 μm) system. The resulting micelle droplet size is then determined using a Malvern 3000 or equivalent instrument.

### Preparation of Oral Product

An oral product is then prepared via the following method:
1. microcrystalline cellulose, sodium chloride and acesulfame K are mixed in a paddle blender as dry ingredients
2. a flavoring agent is then sprayed onto the dry ingredients, and mixed until homogeneous
3. the emulsion prepared above is then sprayed onto the resulting mixture, and mixed until homogeneous
   The resultant oral product has the following components:

| | | Amount (% w/w |
|---|---|---|
| Raw Material | | of product) |
| | Microcrystalline cellulose | 55 |
| | Sodium chloride | 3 |
| | Acesulfame K | 1 |
| | Flavoring Agent | 1 |
| Emulsion | Castor Oil | 3 |
| | Cannabidiol | 1 |
| | Water | 16.056 |
| | Myrj 52 | 3.6 |
| | Lecithin | 3.6 |
| | Glycerine | 12.6 |
| | Sodium benzoate | 0.144 |

The oral product has desirable release and absorption characteristics when placed into the oral cavity of the user.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. An oral product comprising:
(i) a cannabinoid;
(ii) a filler; and
(iii) water;
wherein the water content of the oral product is at least about 10% by weight of the oral product, and
wherein the water activity of the oral product is no greater than about 0.85.

2. An oral product according to claim 1, wherein the water content of the oral product is from about 10% to about 30% by weight of the oral product.

3. An oral product according to claim 1, wherein the filler comprises a cellulose material selected from the group consisting of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, derivatives thereof, and combinations thereof.

4. An oral product according to claim 3, wherein the cellulose material is a derivative of wood pulp fiber.

5. An oral product according to claim 4, wherein the cellulose material is microcrystalline cellulose.

6. An oral product according to claim 1, wherein the filler is present in an amount of at least about 50% by weight of the oral product.

7. An oral product according to claim 1, wherein the filler is present in an amount of from about 55% to about 95% by weight of the oral product.

8. An oral product according to claim 1, wherein the cannabinoid is present in an amount of from about 1% to about 30% by weight of the oral product.

9. An oral product according to claim 1, wherein the cannabinoid is present in an amount of from about 5% to about 15% by weight of the oral product.

10. An oral product according to claim 1, wherein the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

11. An oral product according to claim 1, wherein the cannabinoid comprises cannabidiol.

12. An oral product according to claim 11, wherein the cannabinoid comprises cannabidiol in an amount of at least 98% by weight of the cannabinoid.

13. An oral product according to claim 1, wherein the oral product contains an emulsion comprising a continuous phase and a dispersed phase, wherein the emulsion comprises at least one cannabinoid.

14. An oral product according to claim 13, wherein the oral product further comprises one or more emulsifying agents.

15. An oral product according to claim 13, wherein the emulsion is in the form of a nanoemulsion in which nanoparticles of an oil phase are dispersed in an aqueous phase.

16. An oral product according to claim 15, wherein the cannabinoid is present in the nanoparticles of the oil phase.

17. An oral product according to claim 1, wherein the oral product further comprises at least one additive selected from the group consisting of a flavoring agent, a taste modifier, a preservative, a humectant, a sweetener, a binder, a buffering agent, salt and mixtures thereof.

18. An oral product according to claim 17, wherein the oral product comprises at least one humectant.

19. An oral product according to claim 18, wherein the humectant is selected from the group consisting of glycerine, 1,2-propanediol, 1,3-propanediol, dipropylene glycol, sorbitol, xylitol, maltitol, and mixtures thereof.

20. An oral product according to claim 18, wherein the humectant is present in an amount of from about 0.1% to about 20% by weight of the oral product.

21. An oral product according to claim 1, wherein the oral product comprises salt.

22. An oral product according to claim 1, wherein the oral product is chemically and physically stable for a period of at least 6 months.

23. An oral product according to claim 1, wherein at least 50 wt % of the cannabinoid is released within at most about 60 minutes when placed in the oral cavity of a user.

24. An oral product according to claim 23, wherein at least 30 wt % of the released cannabinoid is absorbed into the oral mucosa within at most about 60 minutes.

25. A pouched oral product comprising a saliva permeable pouch and the oral product of claim 1 incorporated within the pouch, wherein the water activity of the oral product is no greater than about 0.85.

26. A package containing the pouched oral product according to claim 25.

27. A process for preparing an oral product according to claim 1, the process comprising:
(a) providing a cannabinoid, a filler and water, and
(b) contacting the cannabinoid, the filler and the water to form an oral product;
wherein the water activity of the oral product is no greater than about 0.85.
